(19) United States
(12) Patent Application Publication
(10) Pub. No.: US 2008/0215366 A1
(43) Pub. Date: Sep. 4, 2008

(54) ASSET MANAGEMENT SYSTEM AND METHOD

(76) Inventors: Peter Robson, Plymouth, MN (US); Bruce Gilmore, Plymouth, MN (US); Brent Anderson, Plymouth, MN (US); Scott Ellingboe, Plymouth, MN (US); Rajeev Tandon, Plymouth, MN (US); Jami Schupp, Plymouth, MN (US); Gary Long, Plymouth, MN (US); Martin Oiumet, Plymouth, MN (US); Daniel Schwarz, Plymouth, MN (US)

Correspondence Address: JOHN ALEXANDER GALBREATH, 2516 CHESTNUT WOODS CT, REISTERSTOWN, MD, 21136, US

(21) Appl. No.: 12/038,828

(22) Filed: Feb. 28, 2008

Related U.S. Application Data
(60) Provisional application No. 60/892,115, filed on Feb. 28, 2007.

(51) Int. Cl. G06Q 10/00

(52) U.S. Cl. 705/2

(57) ABSTRACT

An asset management system and method is disclosed, useful for managing any asset or collection of assets that need to be identified by unique characteristics. The system comprises three basic modules: a data input module for inputting information about an asset; a “gateway” module that checks the accuracy of the incoming data and corrects it as necessary; and a trading module that allows users to list their assets in various ways and perform various actions, such as selling the assets to other users or otherwise disposing of them, purchasing additional assets, etc.

### CROSS-REFERENCES TO RELATED APPLICATIONS

This application claims priority from, and the benefit of, applicants' provisional U.S. Patent Application No. 60/892,115, filed Feb. 28, 2007 and titled “Asset Management System and Method”.

### BACKGROUND

1. Field of the Invention

The invention is in the field of systems and methods for managing assets, both generally and in particular medical equipment assets.

2. Description of the Related Art

U.S. Pat. No. 6,466,917 issued to Goyal et al. for “Method and Apparatus for Verifying the Identity of A Participant Within An On-Line Auction Environment.”, teaches a method and apparatus for verifying identity of a participant in a network-based transaction facility. The user interface information is provided to the participant via a communications network. The user interface information specifies an identity verification interface for obtaining personal information of the participant. The personal information of the participant is passed to a third party for verification via the communications network. Subsequently, a verification result is received from the third party via the communications network. The verification result is then communicated to the participant via the communications network. In sum, Goyal relates generally to trust and verification of the participants in the auction system, but not the items to be sold or auctioned.

U.S. Pat. No. 6,732,161 issued to Hess et al. for “Information Presentation and Management in an Online Trading Environment.”, teaches a method and apparatus for information presentation and management in an online trading environment. Person-to-person commerce over the Internet is facilitated by providing prospective buyers the ability to quickly preview items for sale. Images are harvested from a plurality of sites based upon user-supplied information. The user-supplied information includes descriptions of items for sale and locations from which images that are to be associated with the items can be retrieved. Thumbnail images are created corresponding to the harvested images and are aggregated onto a web page for presentation at a remote site. The user may submit a query to preview the items. In response to the query, thumbnail images corresponding to items that satisfy the user query are displayed, each of the thumbnail images previously having been created based upon a user-specified image.

U.S. Pat. No. 7,007,076 issued to Hess et. al for “Information Presentation and Management in an Online Trading Environment.”, also teaches and claims a method and apparatus for information presentation and management in an online trading environment. Person-to-person commerce over the Internet is facilitated by providing prospective buyers the ability to quickly preview items for sale. Images are harvested from a plurality of sites based upon user-supplied information. The user-supplied information includes descriptions of items for sale and locations from which images that are to be associated with the items can be retrieved. Thumbnail images are created corresponding to the harvested images and are aggregated onto a web page for presentation at a remote site. According to another aspect of Hess '076, a user may submit a query to preview items for sale. After receiving the query, thumbnail images corresponding to items that satisfy the user query are displayed, each of the thumbnail images previously having been created based upon a user-specified image.

U.S. Pat. No. 5,845,265 issued to Woolston for “Consignment Modes.”, describes a method and apparatus for creating a computerized market for used and collectible goods by use of a plurality of low cost posting terminals and a market maker computer. The market is in a legal framework that establishes a bailee relationship and consignment contract with a purchaser of a good at the market maker computer that allows the purchaser to change the price of the good once the purchaser has purchased the good thereby to allow the purchaser to speculate on the price of collectibles in an electronic market for used goods while assuring the safe and trusted physical possession of a good with a vetted bailee.

U.S. Pat. No. 6,085,176 issued to Woolston for “Method and Apparatus for Using Search Agents to Search Plurality Of Markets for Items.”, describes a similar method and apparatus for creating a computerized market for used and collectible goods by use of low cost posting terminals and a market maker computer in a legal framework that establishes a bailee relationship and consignment contract with a purchaser of a good at the market maker computer that allows the purchaser to change the price of the good once the purchaser has purchased the good thereby to allow the purchaser to speculate on the price of collectibles in an electronic market for used goods while assuring the safe and trusted physical possession of a good with a vetted bailee.

U.S. Pat. No. 6,202,051 issued to Woolston for “Facilitating Internet Commerce through Internetworked Auctions.”, describes auctioning an uniquely identified item (e.g., used goods or collectibles) with a computerized electronic database of data records on the Internet includes creating a data record containing a description of an item, generating an identification code to uniquely identify the item, and scheduling an auction for the item at the computerized database of records. The item is presented for auction to an audience of participants through a worldwide web mapping module executing in conjunction with the computerized database. The data record connotes an ownership interest in the item to a seller participant on the computerized electronic database of data records. The worldwide web mapping module translates information from the data record on the computerized database of records to a hypertext markup language (HTML) format for presentation through the Internet. Bids are received on the item from participants on the Internet through an auction process that executes in conjunction with the computerized database of data records. Auctioning of the item is terminated when the auction process reaches predetermined criteria. The auction participant is notified of the high bid in the auction process. The unique identification code is provided to the auction participant with the high bid to uniquely identify the item.

U.S. Pat. No. 5,664,111 issued to Nahan et al. for “Computerized, multimedia, network, real time, interactive marketing and transactional system.”, teaches a system for art dealers to sell artwork by using a network of computers. The system enables users to store and retrieve images and data pertaining to the artwork and to purchase artwork corresponding to the images.

### SUMMARY OF THE INVENTION

The invention is an asset management system and method, useful for managing any asset or collection of assets that need to be identified by unique characteristics. The invention is applied herein to the management of medical equipment; however, it can also be applied to a variety of other assets, including but not limited to automobile parts, computers and other electronic devices, and even fine art.

The system comprises three basic modules: a data input module for inputting information about an asset; a “gateway” module that checks the accuracy of the incoming data and corrects it as necessary; and a trading module that allows users to list their assets in various ways and perform various actions, such as selling the assets to other users or otherwise disposing of them, purchasing additional assets, etc. Registered members of the trading module have the ability to not only list their owned equipment for sale—i.e., equipment that they themselves own—but also to manage the asset inventory of clients for whom they are a registered agent. For example, a registered agent of a particular hospital can manage that hospital's equipment assets, including offering surplus equipment for sale, donating equipment, buying needed equipment, disposing of obsolete equipment, etc.

The inventive system and method thus provides a valuable set of asset management tools to the user. By way of non-limiting example, the user can easily edit information about his equipment, can easily change the status of a piece of equipment to list it for sale, donate, recycle, or otherwise dispose of it, and can use historical sales to help determine an appropriate current price for a particular piece of equipment. Users can also bundle together assets in a multi-piece lot, for sale together at a collective price. As part of the inventive method, assets are also checked to ensure that they meet various federal, state, and local regulations, and users are checked to ensure that they are “trusted” and authorized to complete transactions concerning the assets.

The system and method of the invention thus provides users with reliability and security concerning the assets listed in the trading module—i.e., users can be confident that the information given for a listed asset is accurate and reliable, so that users know exactly what they are buying.

### DESCRIPTION OF THE DRAWINGS

FIG. 1 shows the system, comprising three broad modules.

FIGS. 2 to 13 show the various screens in the on-line platform of the trading module that are employed by users.

FIG. 14 shows the “Advanced Search” functionality of the system.

FIG. 15 shows the main screen of the administrative site.

FIG. 16 shows the screen used for connecting to the equipment database.

FIG. 17 shows the screen used to upload the equipment data into the staging area.

FIG. 18 shows various information about the staged equipment.

FIG. 19 shows the screen used to make changes to the equipment information.

FIGS. 20 & 21 display all the equipment active on the trading platform.

FIG. 22 displays various information about a particular piece of equipment.

FIG. 23 displays various information about the registered agent associated with a particular piece of equipment.

FIG. 24 shows various information about the active lots.

FIG. 25 displays various information about a selected lot.

FIG. 26 shows the screen that allows the administrator to view all the general members who have signed up to view equipment on the trading platform and make offers for equipment.

FIG. 27 displays new registrations that have not yet been approved or declined by the administrator, as well as active registrations.

FIG. 28 shows the screen that allows the administrator to view previously-added news articles about the system and method that are displayed on the main user page.

FIGS. 29-32 show the JETT.eye PDC/handheld specifications and capabilities.

FIG. 33 shows the select location screen.

FIG. 34 shows the add item screen.

FIG. 35 shows the attach a picture screen.

FIG. 36 shows the edit the image screen.

FIG. 37 shows the edit the location item screen.

FIG. 38 shows examples of system-generated barcodes.

FIG. 39 shows the edit lot information screen.

FIG. 40 shows the select location to transmit screen.

FIGS. 41 & 42 show the data validation process employed by the Gateway Module (FIG. 42 is a continuation of FIG. 41).

FIGS. 43-48 show the operation of various user screens concerning the wish list feature.

FIGS. 49-54 show the operation of various user screens concerning the redistribution feature.

FIGS. 55-59 show the operation of various user screens concerning the donation feature.

### DETAILED DESCRIPTION OF THE INVENTION

The following provides a list of the reference characters used in the drawings:
1. Data input module
2. Gateway Module
3. Trading module
4. Application server
5. Asset Database
6. Inventoried asset list
7. Sub-categories
8. Equipment information
9. Lot equipment information
10. Newly listed inventory
11. Newly listed lots
12. Equipment description
13. Condition indication
14. Make an offer button
15. Watch this item button
16. Send a message icon
17. Inquiry icon
18. Offer amount
19. Offer text box
20. Send offer to owner button
21. Watch this lot button
22. Messages button
23. Watch list button
24. Remove button
25. Documents button
26. Seller agreement button
27. My inventory button
28. Company drop-down menu
29. Location drop-down menu
30. Status drop-down menu
31. Select check box
32. Change status drop-down menu
33. Update button
34. Item number button
35. Edit button
36. My account button
37. Edit profile button
38. Approved button
39. Quick search text box
40. Advanced search button
41. Switch to main site button
42. Upload button
43. Inventory button
44. Moderate button
45. Clients button
46. System button
47. Handheld button
48. Zip file button
49. Registered agent drop-down menu
50. Import button
51. File text box
52. Browse button
53. Load button
54. Staging button
55. Manage inventory button
56. Staged company drop-down menu
57. Staged location drop-down menu
58. Item view button
59. Lot view button
60. Move to production button
61. ID button
62. Agent button
63. Create lot button
64. Lot ID button
65. Members button
66. Registered button
67. Approved button
68. Declined button
69. Active button
70. Inactive button
71. Approve button
72. Decline button
73. Deactivate button
74. Reg. ID button
75. Search button
76. Messages button
77. News button
78. Location display area
79. Select location button
80. Add location button
81. Edit location button
82. Delete location button
83. Exit button
84. Add button
85. Add+Repeat button
86. VF On button
87. Lamp On button
88. Make Pic button
89. Close button
90. Picture display area
91. Delete button
92. Item image pull-down menu
93. Item display area
94. Edit button
95. Transmit button
96. Staging table
97. Production database
98. Add Lot text box
99. Cancel button
100. Update button
101. Lot display area
102. Find button
103. Add new item button
104. Match drop-down menu
105. Save item button
106. Checkbox
107. Add selected items to sourcing request button
108. Sign in button
109. Inventory filter button
110. Item in my organization indicator
111. Add to shopping cart button
112. Donation item indicator
113. Order ID field
114. Found item indicator

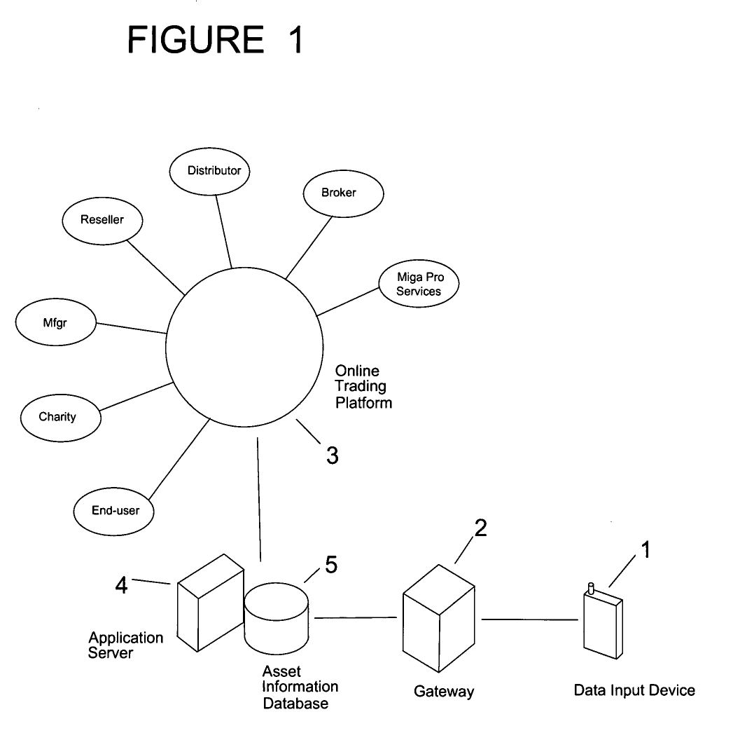

As shown in FIG. 1, the system comprises three broad modules: a data input module 1 for inputting information about an asset; a “gateway” module 2 that checks the accuracy of the incoming data and corrects it as necessary; and a trading module 3 that allows users to list their assets in various ways and trade them with other users.

### Data Input Module

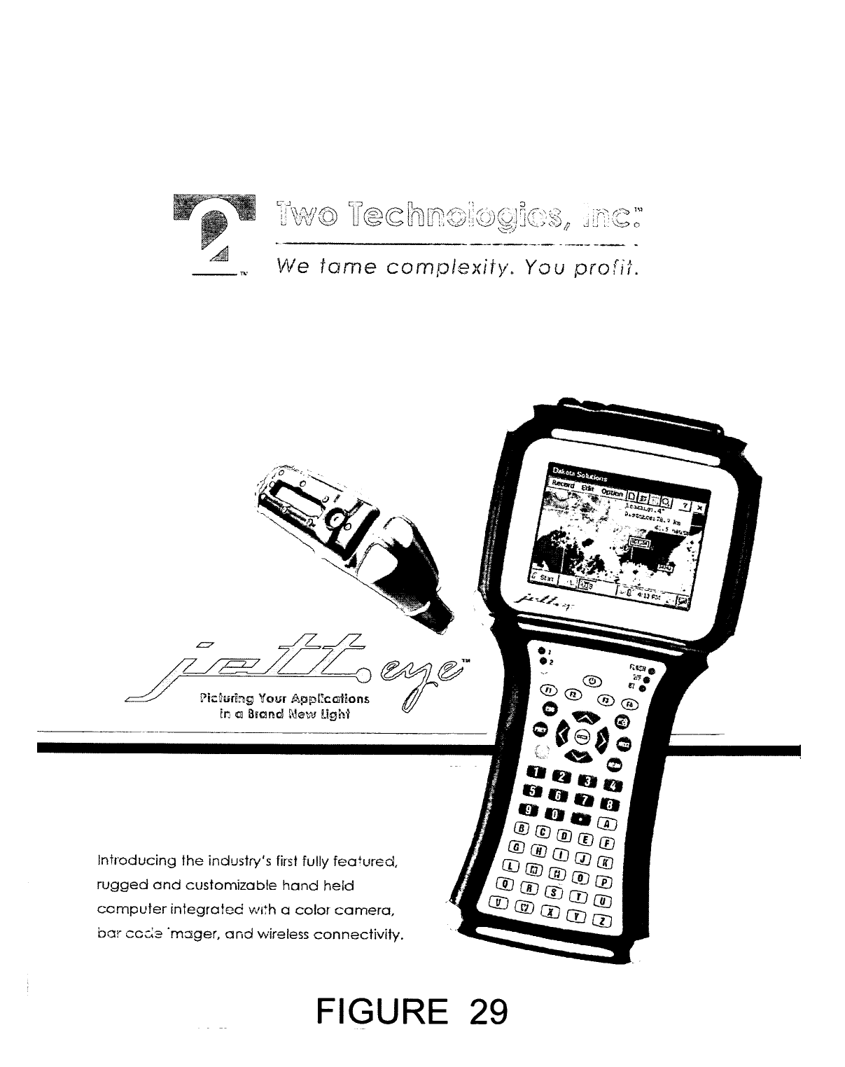

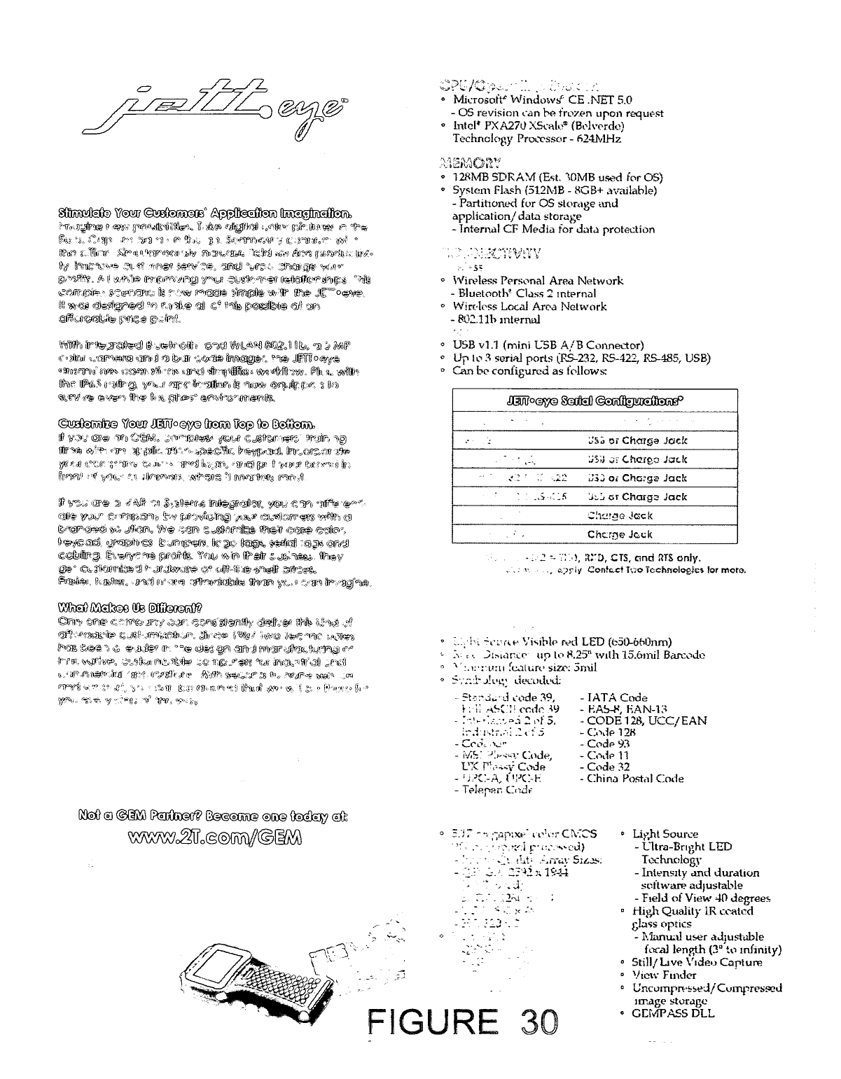

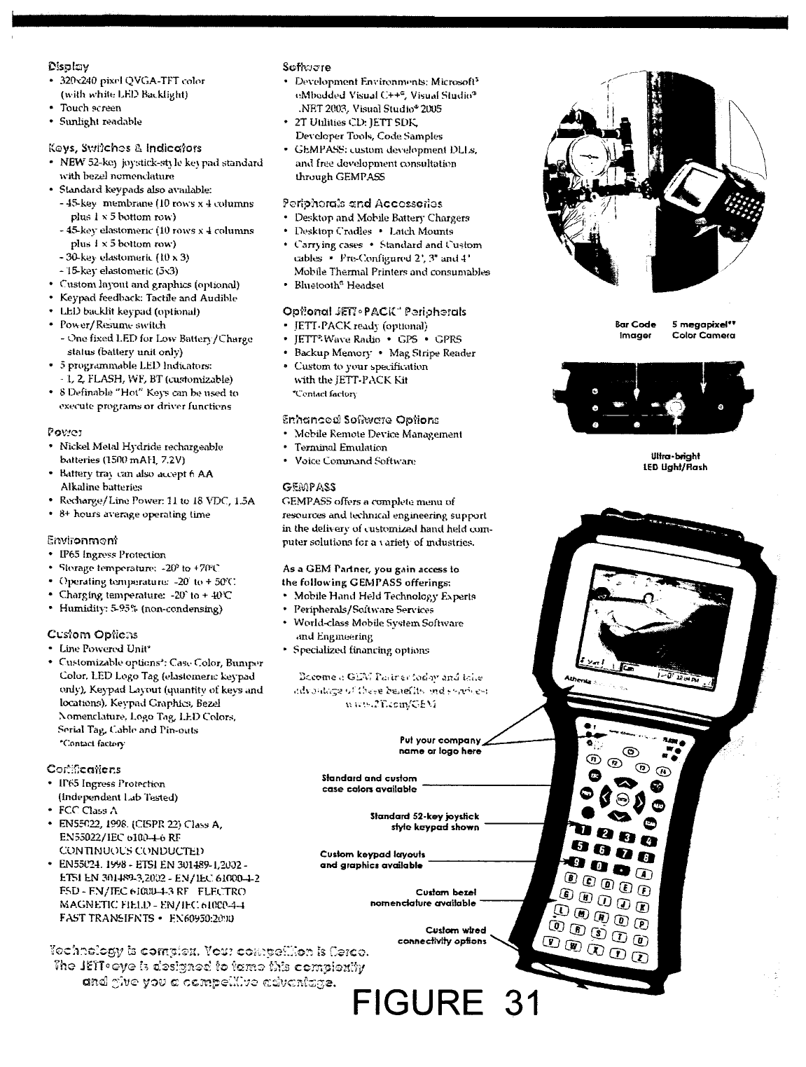

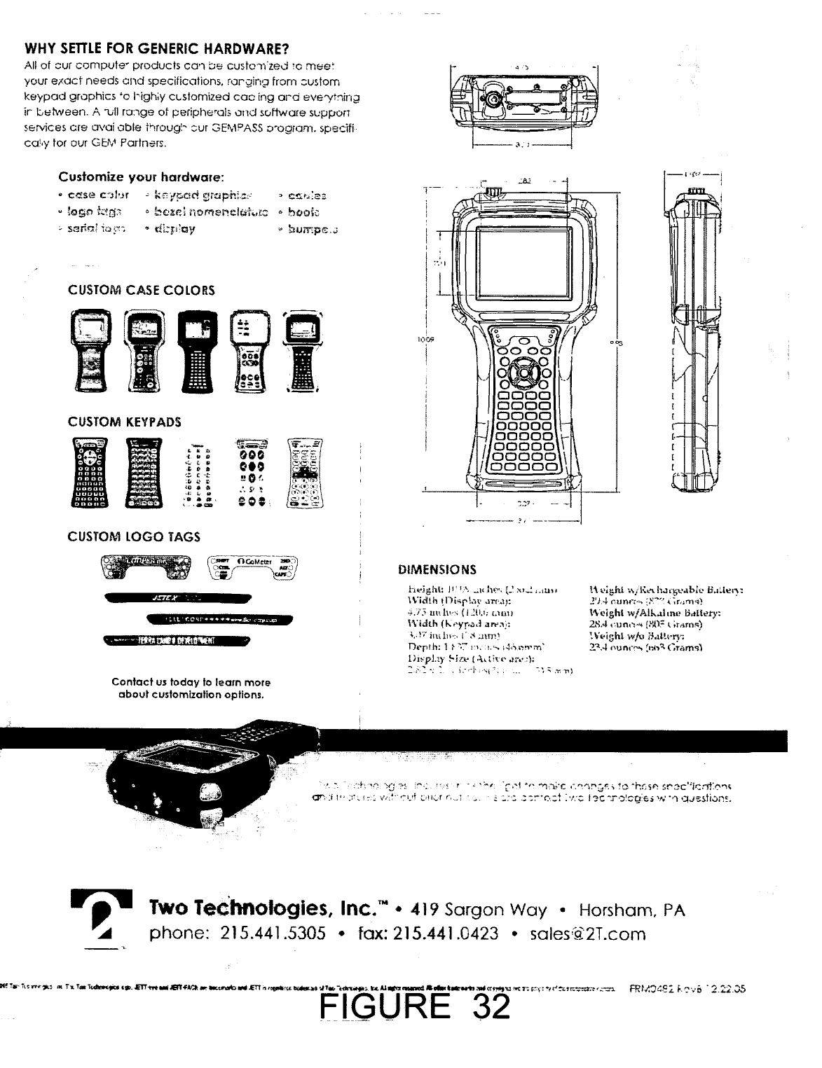

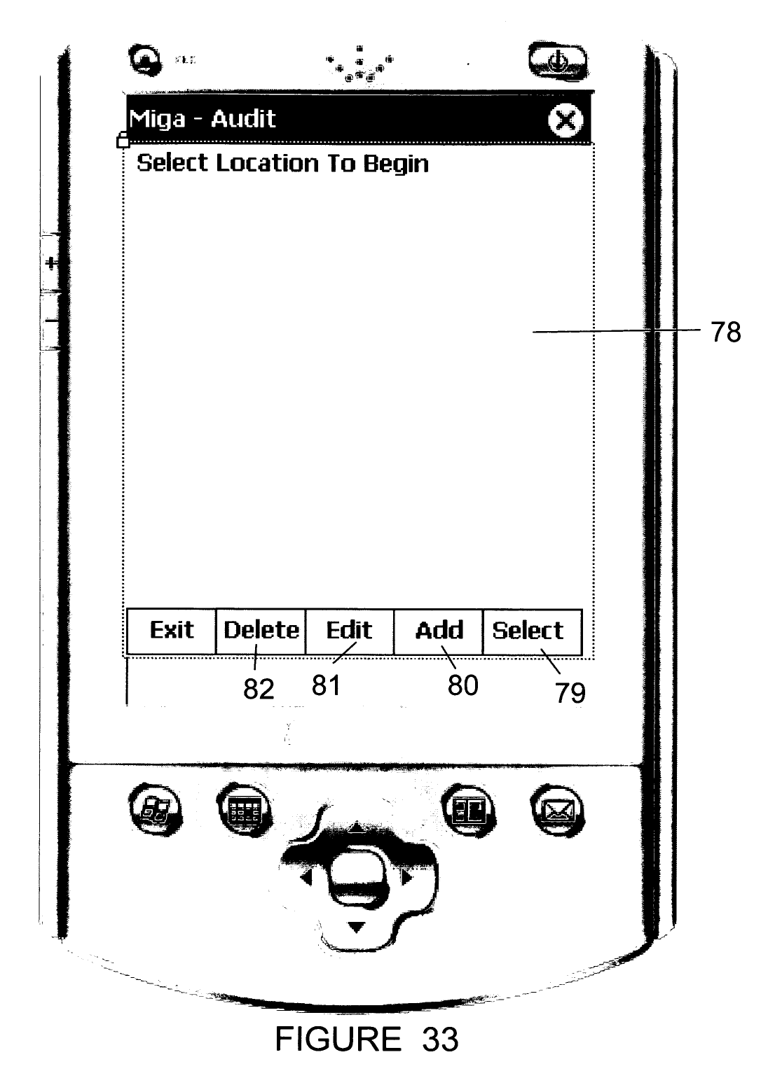

The data input module 1 can take a variety of forms, including but not limited to a desktop computer, laptop computer, a terminal connected to a computer via wired or wireless means, or a handheld data input device (“PDC”). Data input module 1 has a software application residing thereon that controls the input and storage of asset data, or is connected via wired or wireless means to a computer containing said software application. One example of a handheld data input device is the “Pocket PC” shown in FIGS. 33-40. Another example of a handheld data input device includes the “JETT.eye” model manufactured by Two Technologies, Inc. The JETT.eye specifications and capabilities are shown in FIGS. 29-32. Video data (a video image of the asset) may be input via a digital camera on the PDC, audio data (a narrative about the asset) may be input via a microphone on the PDC, and character data may be input via a keyboard/keypad on the PDC. The PDC also has a bar code reader in order to read any bar code tags that are located on the asset (placed there previously, either by an owner or by a user working with the inventive system). The bar code reader can also be used to read a new bar code sticker associated with the inventive system, said bar code sticker being placed on the asset after being “connected” with the asset by the user. The PDC display has touch screen capabilities, so the user can input information about an asset by touching the screen by hand or with a stylus. Although not featured on the JETT.eye PDC shown in FIGS. 29-32, a suitable handheld data input device may also include a radio frequency identification (“RFID”) tag reader, a magnetic tag reader, and a digital video (moving picture) camera. The JETT.eye PDC can communicate the data input into it via a wireless personal area network using Bluetooth communication protocol; a wireless local area network using the 802.11b standard; or a wired USB connection. Serial ports (RS-232, RS-422, RS-485, or USB) can also be configured for communication purposes. It should be understood that other handheld data input devices, such as cell phones, personal data assistants (PDAs), can be adapted to serve as data input module 1. It should also be understood that the data input device could employ any suitable communications protocol, in addition to those listed above.

Using the above communications means, the user of the PDC/handheld first connects via replication to an associated server, in order to prepare the device for information storage. The user then enters information about the asset or assets into the device via the various means summarized above and discussed in detail below (this is also referred to as doing an “audit” of the asset). After the asset information has been entered into the handheld device, the user transmits the information from the device to an associated server via an Internet or other connection, using replication.

The software application that controls the PDC/handheld includes the following functions:

### Application Setup

The application uses replication to obtain the database structure required to perform an audit (enter information about an asset). Since the audit database does not exist on the handheld, after the user starts the application, the application prompts the user to obtain the data structure. The data structures for the following tables are created:

Miga_Location

Miga_Item

Miga_ItemImage

Miga_Lot

Miga_Category

Miga_Category2

Miga_Category3

Miga_Identity

The handheld application does not communicate directly with the asset database; instead it communicates via an IIS server that has the required replication components. The current IIS server url used by the application is: http://migasolutions.net/MobileReplication/sqlcesa30.dll. The address of the asset database server is: 208.101.12.181 (SV1572).

### Select Location

Once the user obtains the data structure used by the program (via the Application Setup function discussed above), they must select a location. The application displays the screen shown in FIG. 33 to the user. The user selects the location where the assets are (and presumably, where the user is or has been) by highlighting a location displayed in location display area 78 and clicking on select location button 79. The application then launches the add item form for that location.

If the location is a new location not previously known to the system, the user clicks add location button 80, which allows the addition of a new location. The user can also change the information about the location by clicking on edit location button 81. Finally, the user can delete a location and all associated information (items, groups, lots, etc.) by clicking on delete location button 82. The user exits the screen shown in FIG. 33 by clicking on exit button 83.

All the subsequent asset-auditing activity performed by the user will be associated with the location the user selected, until the user selects a different location.

### Add Item

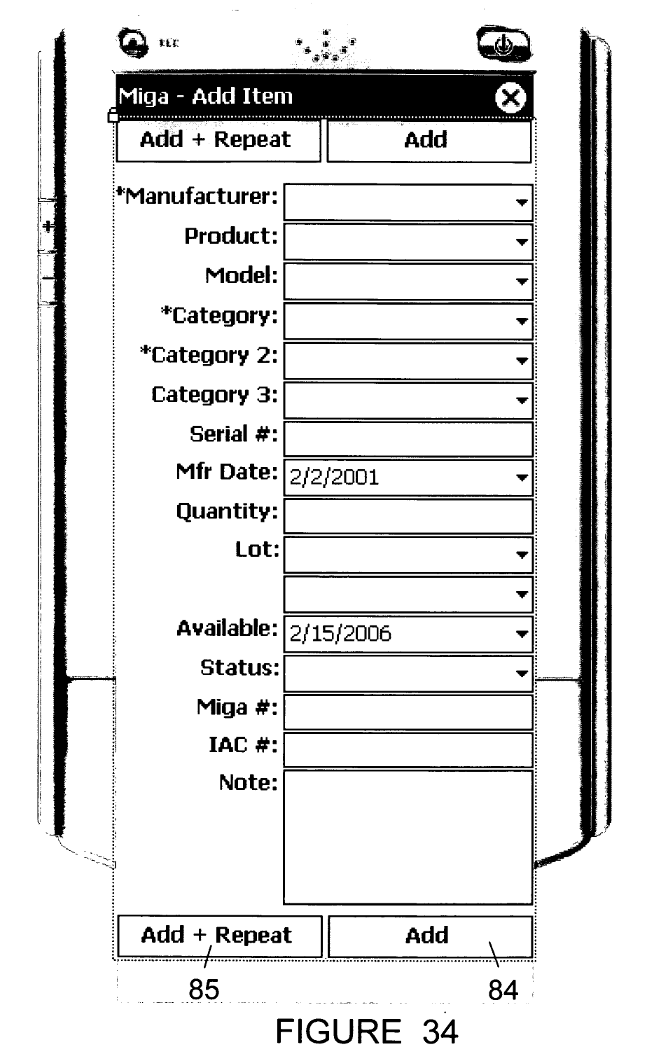

Via this function, shown in FIG. 34, the user adds an item to inventory, such as a piece of equipment or other asset, by entering various information about that asset into the system. A description of the various information follows:
Manufacturer—for example, Siemens or GE.
Product—the product name or a description of what the asset is.
Model—for example, GEAMX 4 (model name and/or number). This can be numeric, alphanumeric, or some other character string.
Category (drop down menu)
   Medical/Surgery equipment.
   Laboratory equip I
   Vivarium equip.
   Other/Misc.
Note: The above categories are for medical equipment, and are provided as an example. It should be understood that various other categories and sub-categories can be created, as appropriate to handle different types of assets.
Category 2 (drop down menu): the user can also select a sub-category in which to place the asset—e.g., a sub-category within medical/surgical equipment.
Category 3 (drop down menu): the user can also select a further sub-category in which to place the asset—e.g., a further sub-category within the sub-category of medical/surgical equipment defined by Category 2.
Serial Number
Manufacturing Date
Quantity
Lot—the user indicates whether the asset should belong to any particular multi-piece lot of assets.
Availability Date—date that the asset is available for pickup.
Status/Recommendation—when initially entered, “Status” indicates the action to be taken concerning the asset, including:
   Sell
   Donate
   Transfer
   To be determined (TBD)
   Recycle
   Trash
Miga #—this is an identifier or internal reference assigned by the maintainer of the system. As discussed below in the Edit Item section, this code can have a barcode associated with it. In other words, the Miga code can simply be an alphanumeric code that is not associated with a barcode, or it can be the alphanumeric equivalent of a barcode associated with the asset.
Internal Asset Control (IAC) #—this is a seller specific identifier or seller internal reference. As discussed below in the Edit Item section, this code can have a barcode associated with it. In other words, the seller code can simply be an alphanumeric code that is not associated with a barcode, or it can be the alphanumeric equivalent of a barcode associated with the asset.
Note—the user can enter a textual description of the asset, or record any other notes concerning the asset.

After the user has completed entry of the asset information, the user clicks on add button 84, thus adding the asset and all its associated information to the database. After the addition of an item, the auditor is prompted to take a picture, and this action is further described below. Optionally, the user can click on add+repeat button 85 in order to avoid some data entry for similar items. The add+repeat function allows the user to save time when entering multiple similar items. When this function is selected, for the current item the PDC will automatically enter the information from the previous item, with the exception of seller code and serial number. The PDC application will prompt the user, signaling an error, if an attempt is made to save data that is an exact match of previously saved data.

### Attach a Picture

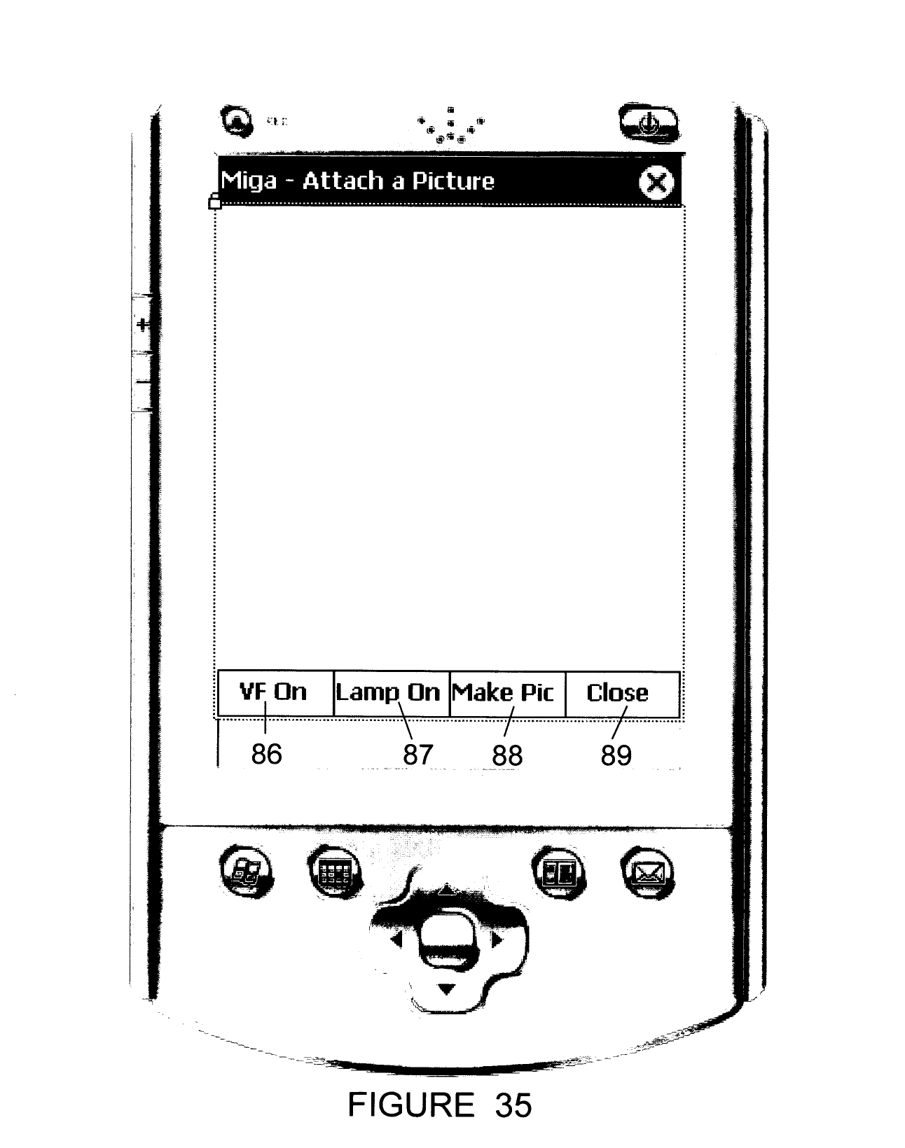

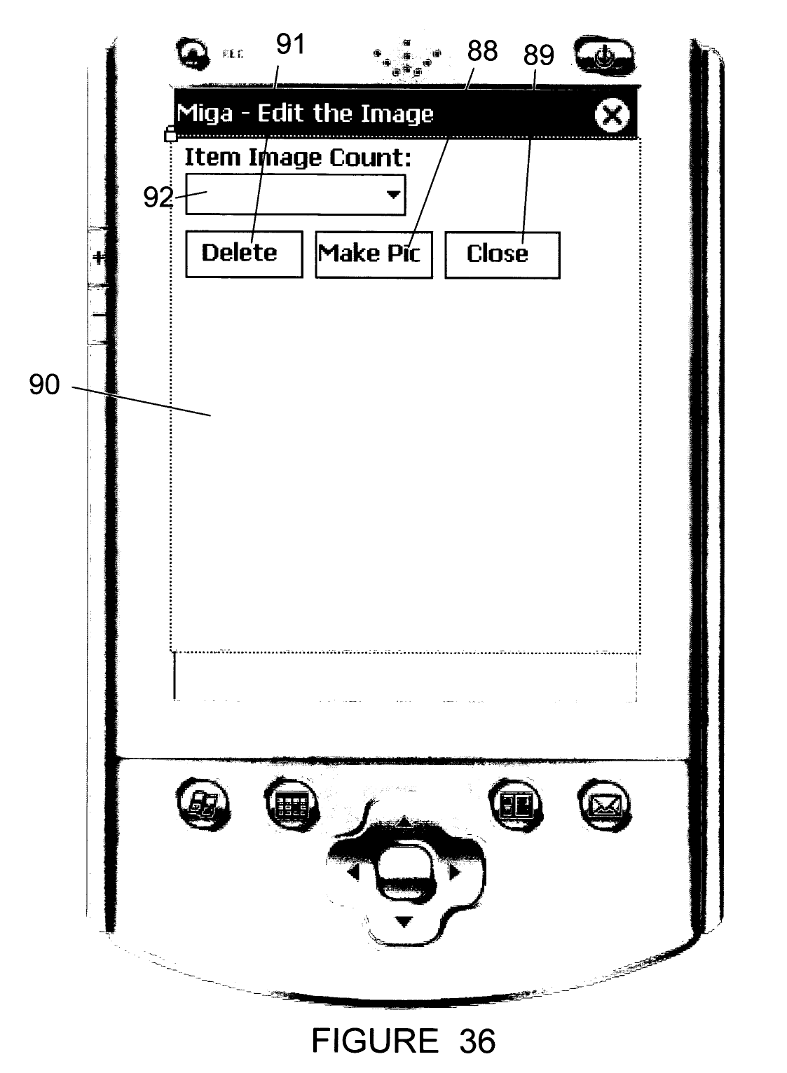

When the user answers yes to the “do you want to take a picture” prompt in the Add Item screen, the screen shown in FIG. 35 is displayed. To take a picture of the asset, the user first clicks VF On button 86, and the screen “opens” into a viewfinder. Alternatively, VF On button 86 can control other viewfinder means. If desired, the user clicks Lamp On button 87, which provides light for flash functionality. The user then clicks Make Pic button 88, and the PDC takes a picture and opens the Edit Image screen shown in FIG. 36 and further described below. To close the screen, the user clicks Close button 89. It should be understood that moving picture video images can also be entered via the PDC, via a screen with similar functionality. It should also be understood that the user can change the size or resolution of the image being taken by the device, via various Image Setting screens.

### Edit the Image

After a picture has been taken, the Edit the Image screen shown in FIG. 36 automatically opens, and the last picture taken is displayed in picture display area 90. The entire picture can be shown, or alternatively, the image can be slightly cropped. The user can then delete the picture by clicking on Delete button 91, take another picture of the item by clicking on Make Pic button 88, or select another picture that has already been taken of the item by clicking on item image pull-down menu 92.

### Location Item

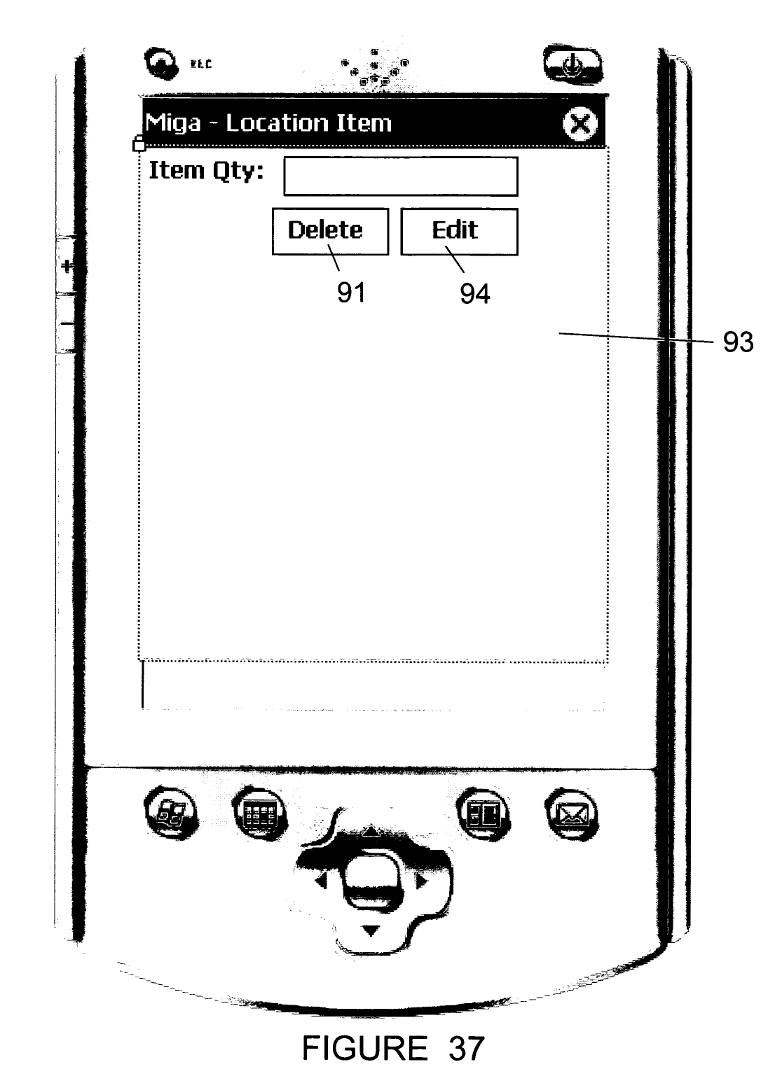

Via the screen shown in FIG. 37, a complete list of all the items that have been entered is displayed in item display area 93. The user can select an item for further editing by clicking on Edit button 94, or delete the item by clicking on Delete button 91.

### Edit Item

Using this function, which employs a screen similar to the Add Item screen shown in FIG. 34, the user can change item information or delete the item.

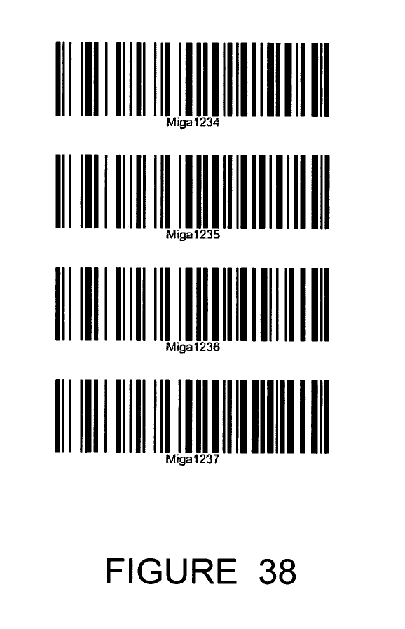

As discussed earlier, via this screen the user or a subsequent user can also scan a bar code, or view information stored in the system for a previously-entered bar-coded item. To scan a barcode, the user activates the scanner by pressing the scanning button on the PDC/handheld. The built-in scanner on the PDC/handheld then scans the barcode and if the barcode starts with “Miga”, the application enters the barcode into the Miga Code field in the Add Item screen shown in FIG. 34. See FIG. 38 for examples of “Miga” barcodes. If the barcode does not start with “Miga” (i.e., it is a barcode not assigned by the user, but rather by the manufacturer, seller, etc.), then the application enters the barcode into the Seller Code field in the Add Item screen shown in FIG. 34. The Seller Code field will accept most barcodes. Said another way, when a barcode is scanned, if it begins with “Miga”, it is used to populate the Miga Code field; otherwise the Seller Code field is populated.

Regarding bar codes, the user can also scan a new barcode sticker and then associate the barcode to the asset. In the discussion above, such a new bar code would be considered a “Miga” barcode assigned by the user. This function allows a person subsequently working with the asset to scan the barcode and immediately know all the relevant information associated with that asset.

### Edit Lot Information

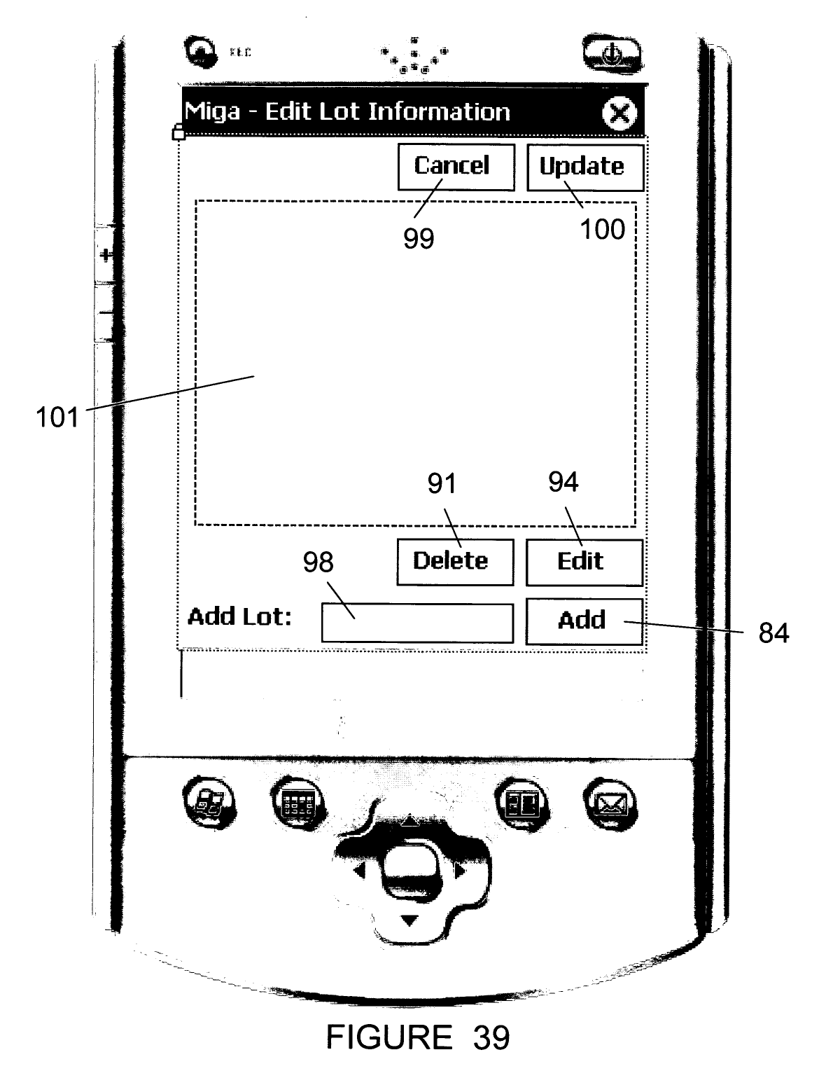

As discussed earlier, the user can create lots—multi-piece collections of assets that are bundled together and sold for one collective price—and assign various assets to these lots. Using the Edit Lot function accessed via the screen shown in FIG. 39, the user sees a list of all the current lots, including their descriptions. The user can edit information for a particular lot, including adding and deleting various items to and from that lot, by highlighting the lot in the displayed list and clicking on Edit button 94. The user can also delete a particular lot by highlighting the lot in the displayed list and clicking on Delete button 91. The user can also add a new lot by clicking on Add button 84, and filling in the name/description of the new lot in Add Lot text box 98. The application verifies that the lot description is unique, and has not already been used. After a new lot has been added, the user can edit the information for that lot, including adding new items to the lot, by highlighting the lot in the displayed list and clicking on Edit button 94 as described above. Finally, clicking on Update button 100 saves any changes the user has made to the lot list or lot information. Clicking on Cancel button 99 while in the middle of making changes (i.e., before clicking on update) will cancel those changes.

### Select Location to Transmit

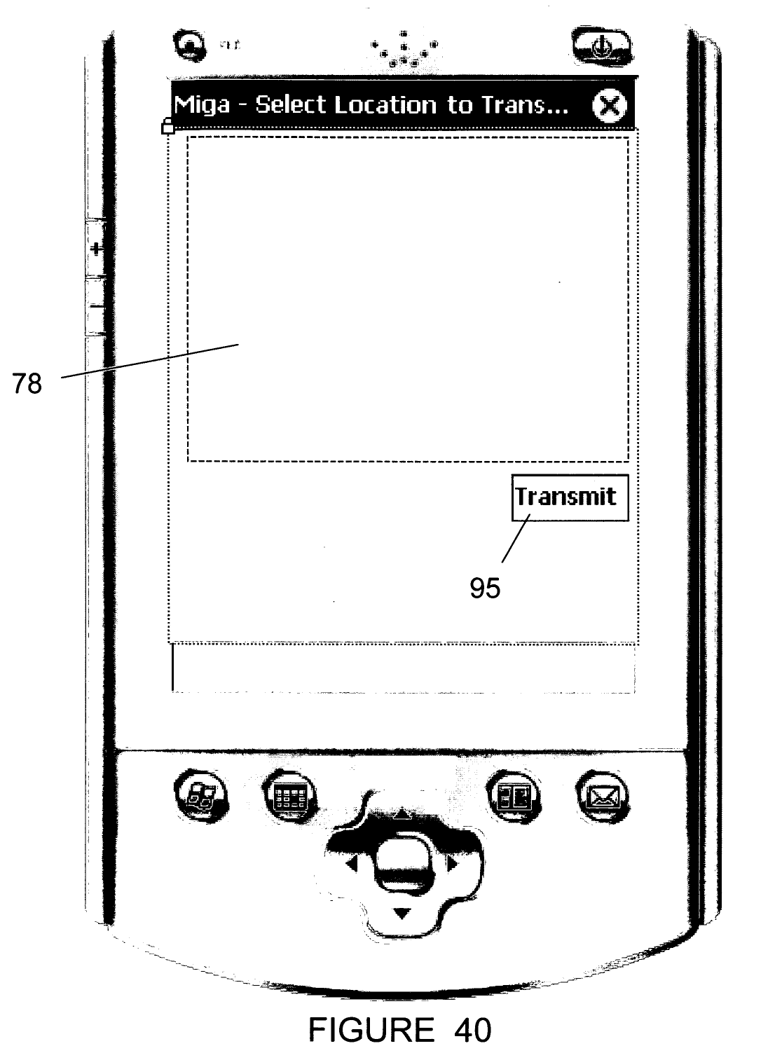

Using this function accessed via the screen shown in FIG. 40, the user can transmit the information he has entered about the items, including images, barcodes, etc., to servers that are part of the inventive system. The user first selects a location to transmit (locations are displayed in location display area 78), then clicks on Transmit button 95 to transmit information from the handheld device to the asset (production) database using database replication. The user is informed of the success or failure of the transmittal process. A transmission file is created for each location that was entered.

It should be noted here that the asset data can be thought of as comprising a plurality of characteristics of a particular asset—for example, a photograph or other visual representation of the asset, a description of the asset, a barcode associated with the asset, etc. To maintain the accuracy and integrity of the data as it is used further by the system, it is important that the entered characteristics be linked together—for example, that the photograph of an asset be linked to its description, serial number, model, manufacturing date, etc. And the sooner this is done, the better. Therefore, in the system and method of the invention, the data input module links at least two of the plurality of characteristics of a particular asset together into a single combined data record for that asset, before transmitting the asset data to the system.

### Gateway Module

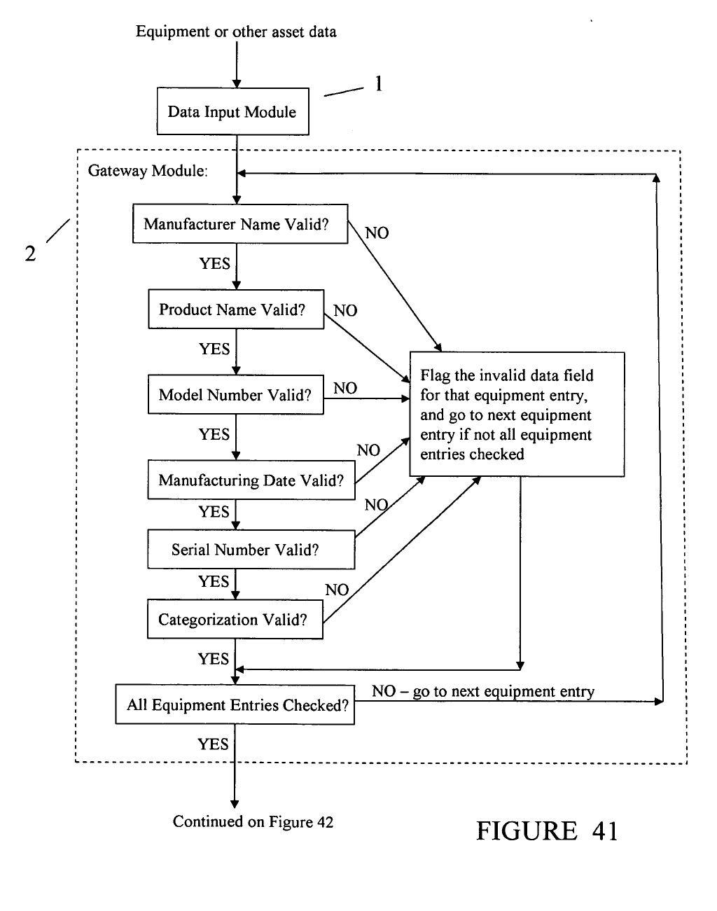

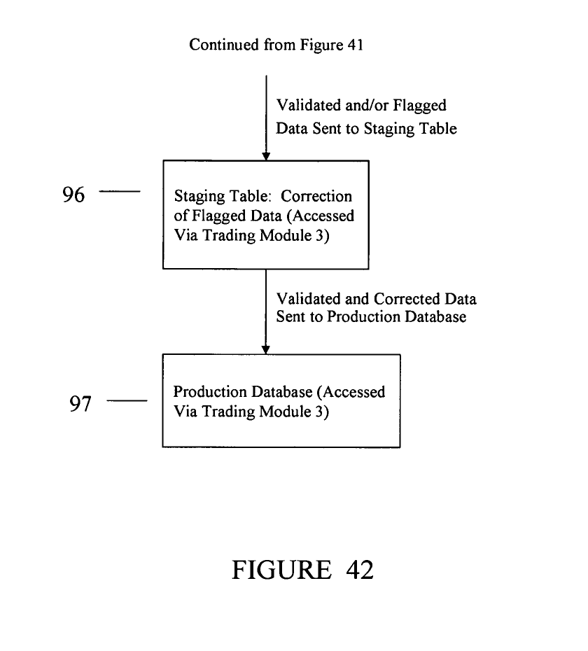

Gateway module 2 is a software application that receives the data that is entered into the system via data input module 1 (an example of which is the handheld device/PDC also referred to in the description, and checks that data for validity. Gateway module 2 can reside on the same server that runs the trading module application described below, or it can reside on a separate server or computer. As shown in FIG. 41, gateway module 2 validates the data it receives from data input module 1 by first checking the validity of the manufacturer name associated with the first equipment entry against a database of valid manufacturer names. As shown in FIG. 41, the validation process is sequential or “tiered”—that is, if gateway module 2 finds the manufacturer name to be valid, it then checks the product name. If it finds the product name to be valid, then it checks the model number, then the manufacturing date, and then the categorization of the asset.

For example, “Lumisys” is a valid manufacturer name denoting Lumisys, Inc. of Sunnydale, Calif. If the handheld/PDC user incorrectly input the manufacturer name as “Lumisis” or “Lumissys”, then gateway module 2 flags as incorrect the manufacturer data field in that particular equipment entry, and goes on to the next equipment entry. If the manufacturer name was entered correctly and thus matches a manufacturer name in the system database, then gateway module 2 goes on to check the product name.

As an aside, the manufacturer name database can be internally-generated, i.e., composed of all the manufacturer names associated with previously validated equipment entries, or alternatively it can comprise a list of manufacturer names obtained from a source outside the system that is either integrated into the system or accessed by the system during the validation process.

Now, when validating the product name, the system already knows the manufacturer from the previous validation step. Since (carrying on with the example discussed above) there are only certain product names used for products from the manufacturer Lumisys, and the system has this product name information either in its internally-generated database of previously validated equipment entries, or in a database obtained from a source outside the system that is either integrated into the system or accessed by the system during the validation process, gateway module 2 can thus check the validity of the product name that was input via data input module 1. For example, “Lumiscan” is one product name used by the manufacturer Lumisys. If the handheld/PDC user entered “Lumscan”, or product name associated not with Lumisys but rather with another manufacturer, then gateway module 2 flags as incorrect the product name field in that particular equipment entry, and goes on to the next equipment entry. If the product name was entered correctly and thus matches a valid product name for the manufacturer Lumisys in the system database, then gateway module 2 goes on to check the model number.

When validating the model number, the system already knows the manufacturer and product name from the previous validation steps. Since there are only certain model numbers used for Lumiscan products from the manufacturer Lumisys, and the system has this model number information either in its internally-generated database of previously validated equipment entries, or in a database obtained from a source outside the system that is either integrated into the system or accessed by the system during the validation process, gateway module 2 can thus check the validity of the model number that was input via data input module 1. For example, “LS20”, “LS50”, “LS75”, and “LS85” are model numbers associated with the “Lumiscan” line of products. If the handheld/PDC user entered “LS100”, or another model number not associated with the Lumiscan product line, then gateway module 2 flags as incorrect the model number field in that particular equipment entry, and goes on to the next equipment entry. If the model number was entered correctly and thus matches a valid model number for the Lumiscan product line in the system database, then gateway module 2 goes on to check the manufacturing date.

When validating the manufacturing date, the system already knows the manufacturer, product name, and model number from the previous validation steps. It then compares the manufacturing date against a database of valid manufacturing date ranges for that particular manufacturer/product/model number. Gateway module 2 obtains this valid manufacturing date range information either from its internally-generated database of previously validated equipment entries, or from a database obtained from a source outside the system that is either integrated into the system or accessed by the system during the validation process. For example, if that manufacturing date range database indicates that Lumisys Lumiscan LS75's were only manufactured from April 1999 through December 2002, and the handheld/PDC user input a manufacturing date of December 2003, then gateway module 2 determines that the manufacturing date was incorrectly input, flags the manufacturing date field in that particular equipment entry as incorrect, and goes on to the next equipment entry. If the model number was entered correctly and is thus within a valid manufacturing date range for Lumiscan LS75's in the system database, then gateway module 2 goes on to check the serial number of the equipment.

Gateway module 2 checks the serial number in a similar manner as the other data—i.e., for a given manufacturer, product, model, and manufacturing date, there are associated correct serial numbers, or a range of correct serial numbers that were assigned to products manufactured on that day. If the serial number was entered correctly and thus matches a serial number in the system database or is within a range of correct serial numbers in the system database, then gateway module 2 goes on to check the categorization of the equipment.

The categorization check is driven primarily by product name and model number, both of which the system already knows from the previous validation steps. Since there are only certain valid category names for Lumiscan LS75's—for example, one such valid category is “Imaging”—and the system has this valid category name information either in its internally-generated database of previously validated equipment entries, or in a database obtained from a source outside the system that is either integrated into the system or accessed by the system during the validation process, gateway module 2 can thus check the validity of the category information that was input via data input module 1. If the handheld/PDC user entered “Cardiology”, or another category not associated with Lumiscan LS75's, then gateway module 2 flags as incorrect the category field in that particular equipment entry, and goes on to the next equipment entry. If the category was entered correctly and thus matches a valid category for Lumiscan LS75's product line in the system database, then gateway module 2 goes on to similarly check the Category 2 and Category 3 data fields (these are subcategories, such as the subcategory “Digitizer” under the broader category “Imaging”).

It should be understood that the above description is illustrative of the sequential data checking process of the invention, and that the order of the data checking steps can be different. For example, the serial number checking step can be done earlier in the process, and can even be checked first. Similarly, a particular data checking step or steps can be omitted from or added to the sequential data checking process, as when the Gateway Module is used to check the accuracy of data entered by a trading module user concerning a particular asset that the user desires but that is not currently available (see the Wishlist section below).

As shown in FIG. 42 (which is a continuation of FIG. 41 at point “A”), after checking all the equipment entries, gateway module 2 sends the validated and/or flagged data to staging table 96. Staging table 96 is essentially a database where the validated and/or flagged data are stored, so that the flagged data fields can be corrected either manually (though human intervention) or automatically, via a correction application with artificial intelligence (AI) capability. The staging table can be accessed by an administrative user of trading module 3, as described below. The administrative user/system administrator, via various screens run by the trading module application, can see which data fields have been flagged by gateway module 2 for each equipment entry in the staging table, and thus need to be corrected. Using his/her own knowledge of the particular assets involved, or another data source, the administrative user corrects the invalid data fields.

It can be appreciated that once the administrative user corrects, for example, an invalid manufacturer name for a particular equipment entry, the validation process can then be resumed for the data fields “below” the manufacturer name in the validation sequence—that is, product name, model number, etc. The validation process can be resumed either by running the data back through gateway module 2 or by the administrative user running the validation process directly from staging table 96.

Automatic correction of the invalid data fields can also be done, either within the validation process of gateway module 2 or while the data are at staging table 96, by a software application with AI capability. The AI automatic correction logic is based on the system's knowledge of valid information already in its databases or available from an outside source. For example, if the product name entered via the handheld/PDC were incorrect but other data fields like manufacturer name and model number were consistent with each other, then the system would automatically make the judgment that only the product name needed correction, and would replace the incorrect product name with the correct one for that manufacturer and model number, obtained from its own databases or from an outside source.

Continuing in FIG. 42, once the flagged data fields have been corrected, and the data entered via data input module 1 have been fully validated, then the data is uploaded to production database 97. The uploading can either be triggered by the administrative user/system administrator, or it can occur automatically once the data for a given equipment entry, or a set of equipment entries, have been fully validated. Once the equipment data is in production database 97, it can be accessed by users of trading platform 3, as discussed below.

In addition to checking the validity of various data entered via data input module 1, the system also performs other valuable checks on the equipment or other assets. For example, many pieces of medical equipment—such as sonogram/ultrasound machines, have on-board memory that contains patient information. This patient information is private, and the Health Insurance Portability and Accountability Act (HIPAA) requires that patient information stored on the equipment be erased before the equipment can be legally sold or otherwise transferred or disposed of.

The system handles this requirement by checking the equipment manufacturer/product name/model number against an internal or external database of known equipment with the capability of storing patient information on board. If a particular piece of equipment does have this capability, then the system alerts the system administrator that the equipment cannot be sold, transferred, or disposed of without the patient information first being erased, or the system automatically places a “hold” on that equipment until the patient information issue is addressed.

Alternatively, the “Add Item” screen of the handheld/PDC, shown in FIG. 34, can have an additional two check boxes, headed by “Patient Data Erased, If Applicable?” and labeled “Yes” and “No”. If the handheld/PDC user checks yes, then the equipment can be sold, etc. after it passes through validation and is uploaded to the production database. If the handheld/PDC user checks no or does not check a box, then a hold is placed on the equipment, and it cannot be sold, etc. after it passes through validation and is uploaded to the production database.

### Trading Module

Trading module 3 is a software application suitable for operation on the Internet or via other hardware means. In its Internet form, trading module 3 is an on-line platform supported via an application server 4 with access to an asset database 5. Asset database 5 has stored therein all the information relating to the assets which will be traded by users using trading module 3. The users of trading module 3 can be, by way of non-limiting example, a broker, distributor, or reseller of the assets; a manufacturer of the assets; a charity seeking to secure funds via donations of assets; and of course end-users seeking to obtain assets for their own use or conversely, dispose of their own assets.

### User Realm

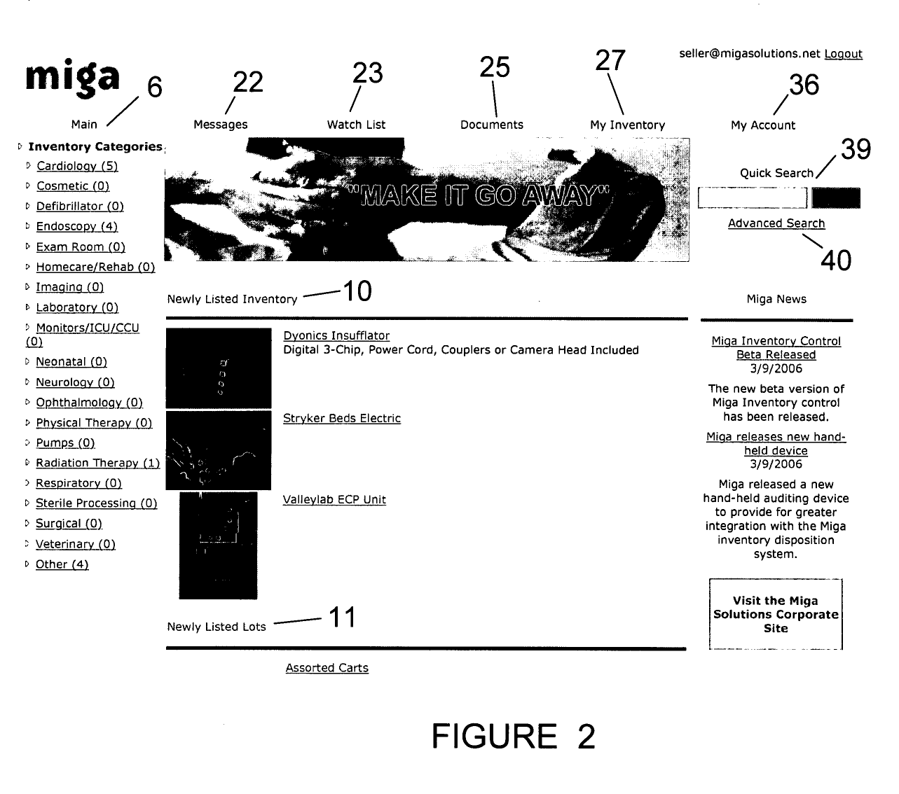

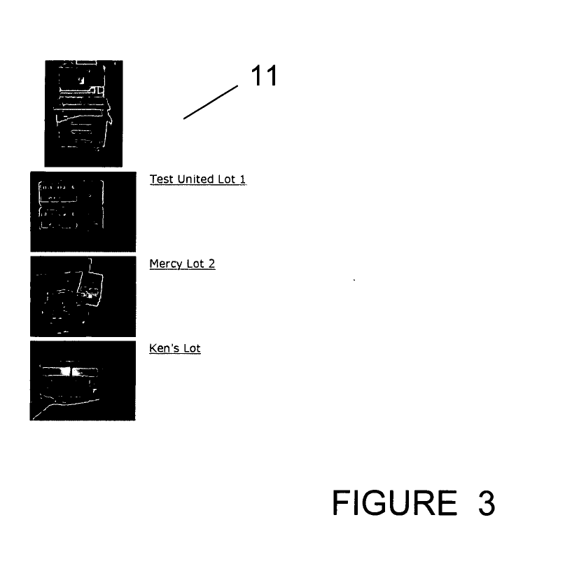

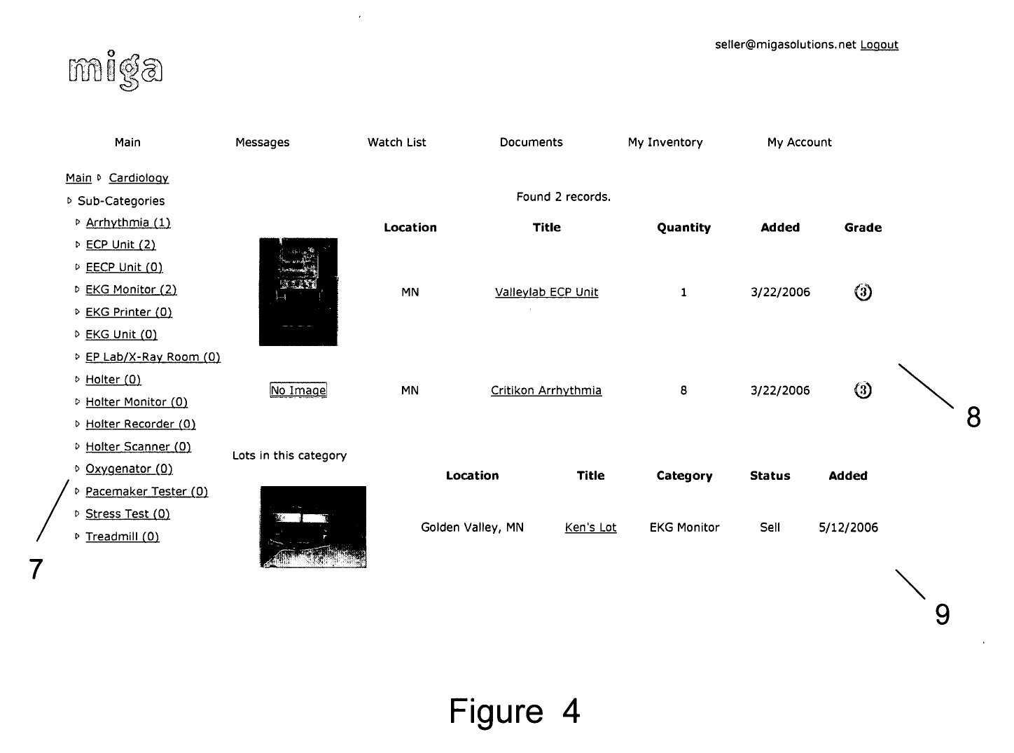

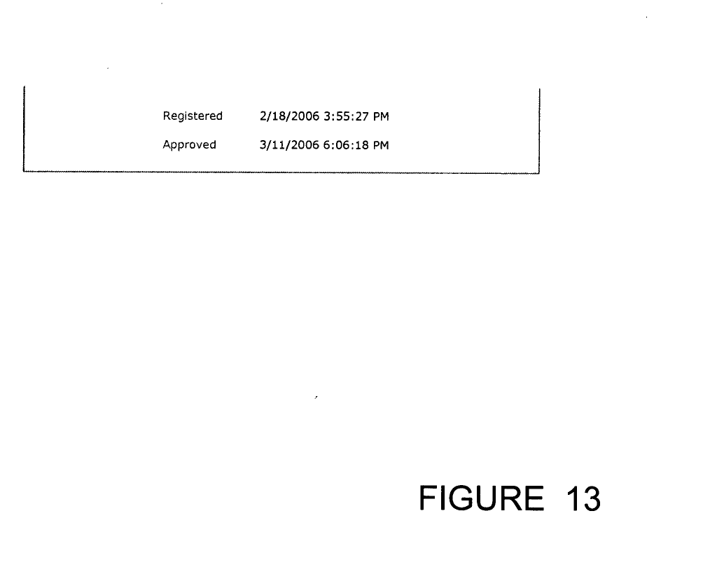

The various screens in the on-line platform of trading module 3 that are employed by users are shown in FIGS. 2 to 13. After logging into the platform, the user is presented with the screens shown in FIGS. 2 & 3. FIGS. 2 & 3 illustrate a main or home page containing an inventoried asset listing 6 (in this example, medical equipment assets) separated into various categories such as Cardiology, Cosmetic, Defibrillator, etc. The number of assets—here, the number of pieces of medical equipment—is shown in parentheses next to the category title. The user can click on each inventory category and thus access the more detailed screen shown in FIG. 4, wherein sub-categories 7 are shown, corresponding to the main category selected. For example, it can be seen that the five pieces of cardiology equipment indicated by FIG. 2 are made up of one piece of arrhythmia equipment, two ECP units, and two EKG monitors.

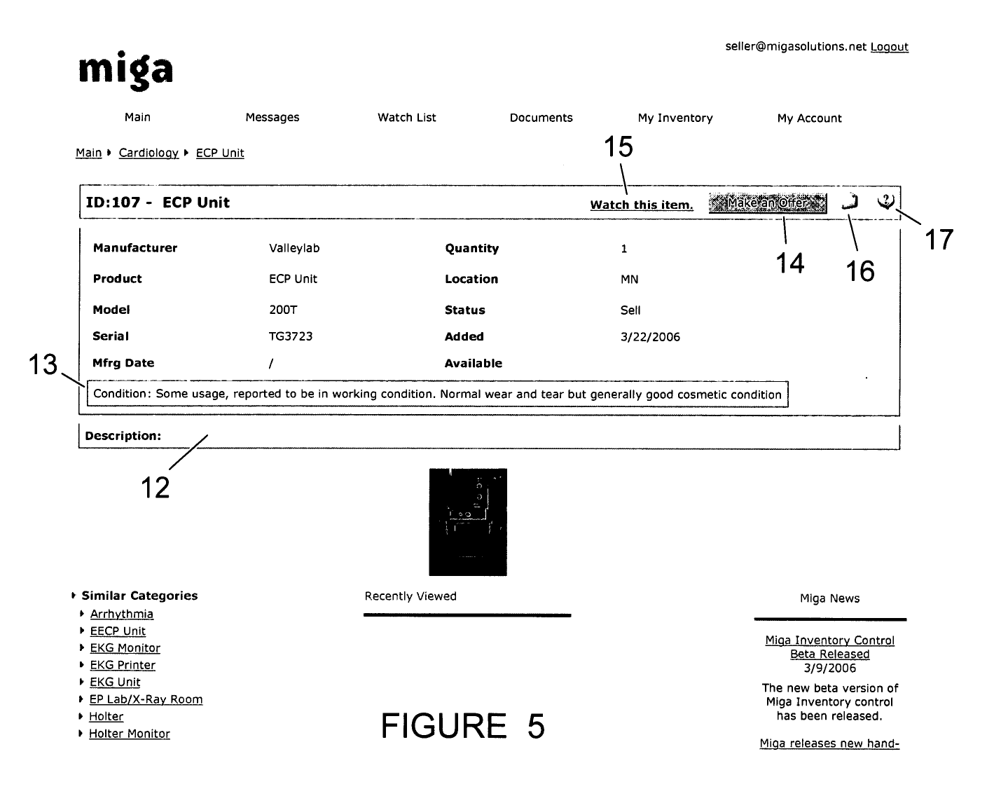

FIG. 4 also contains further equipment information 8 for each piece of equipment in the cardiology category, including its location, title, quantity, date added to the inventory, a “grade” corresponding to the condition of the equipment, and if available, a picture of the equipment. FIG. 4 also shows any multi-piece “lots” of equipment that include cardiology equipment pieces, and shows information about each lot such as its location, title, category/sub-category, status, a representative picture, and the date the lot was added to the inventory. The status assigned to an item indicates the action to be taken concerning it, or the action that has been taken concerning it. Status examples include “Sell”, “Donate”, “Transfer”, “Recycle”, “Trash”, and “To be determined”, as well as “Sold”, “Donated”, “Transferred”, “Recycled”, “Trashed”, and “Removed”. The user can click on the title of each piece of equipment or lot, or its picture if available, to access further information about the assets as shown in FIG. 5.

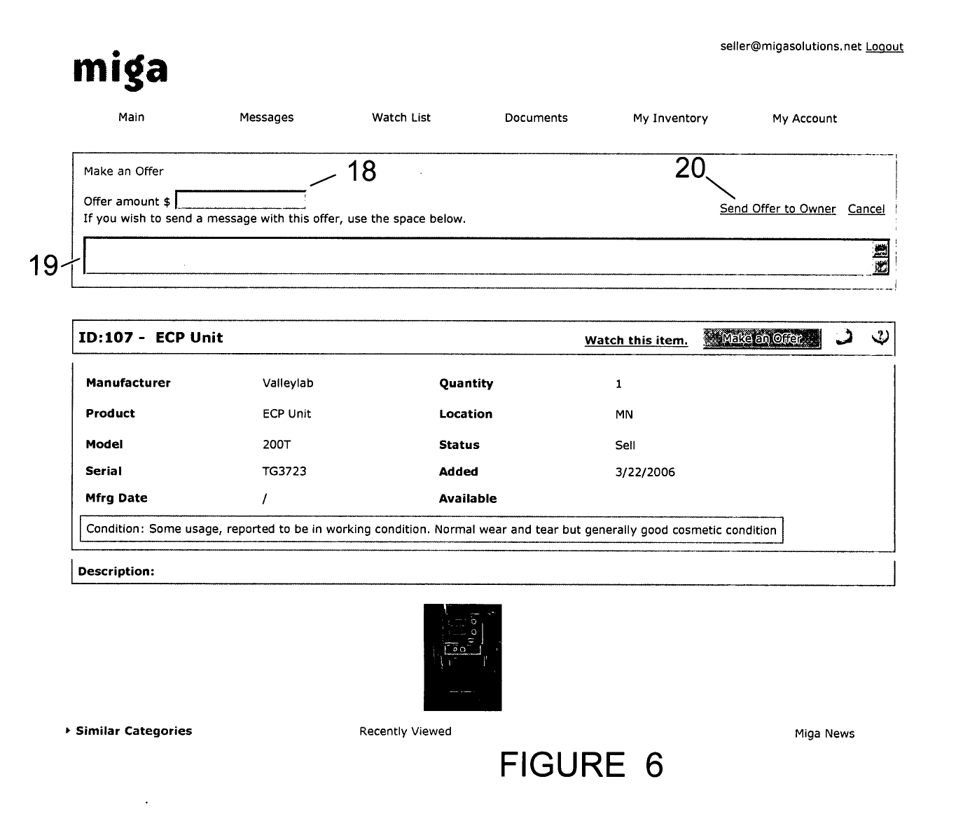

The screen shown in FIG. 5 is the primary screen used to list information about a particular piece of equipment. For example, for the Valleylab ECP unit first shown in FIG. 4, FIG. 5 lists the manufacturer, product, model number, serial number, manufacturing date, quantity, location, status, the date the equipment was added to the inventory, and its availability. A description of the equipment 12, if available, and a condition indication 13 are also shown. From this screen, the user can offer to buy the equipment by clicking on “Make an Offer” button 14, which sends the user to the screen shown in FIG. 6. In FIG. 6, the user fills in an offer amount 18 and can also optionally send a message to the asset owner by filling in the offer text box 19. The user then clicks on the “Send Offer to Owner” button 20 to transmit his offer to the asset owner. It should be understood, here and throughout this application, that an agent or representative may act for the asset owner. A copy of the offer message is also sent to the system administrator. Subsequent negotiations between the seller and buyer can either be done outside the inventive system, or via an “e-commerce engine” internal to the system.

Returning to FIG. 5, the user can also add the item to his watch list by clicking on “Watch this item” button 15. Additionally, the user can send a message via email or other means to the asset owner, without putting in an offer, by clicking on the “Send a Message” icon 16, and can also send an inquiry to the platform administrator by clicking on the “Inquiry” icon 17.

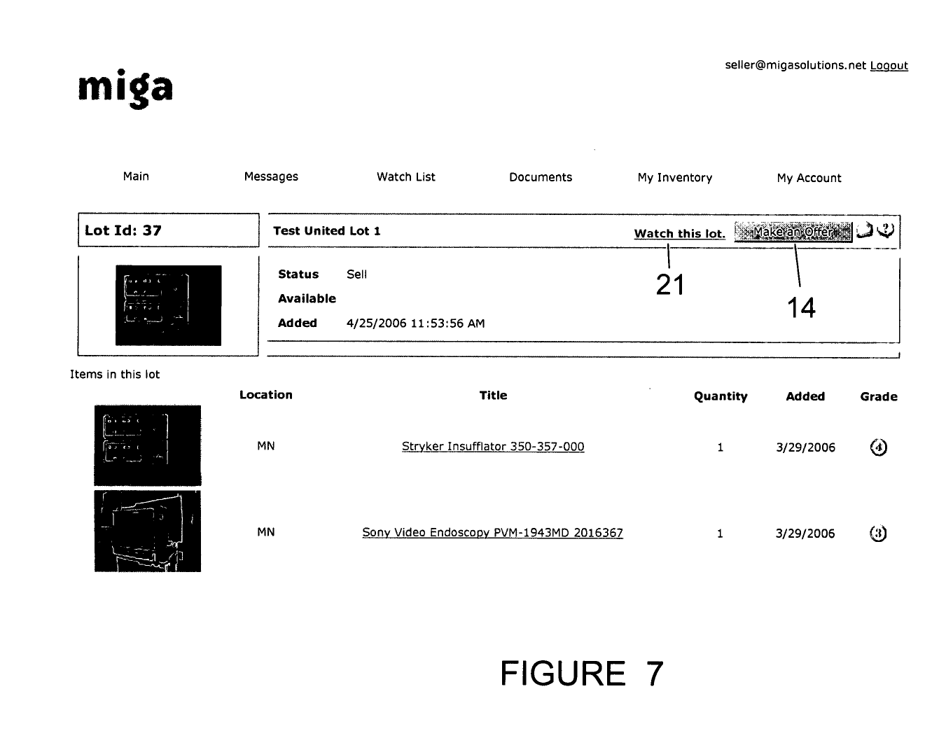

Turning back now to FIGS. 2 & 3, the main screen also shows newly listed inventory 10, and newly listed multi-piece lots 11. The user can click on the title or the picture of a newly listed piece of equipment, and will be sent to a primary information screen similar to that shown in FIG. 5—although this primary information screen will of course show information corresponding to the requested piece of equipment, and not the specific piece of equipment shown in FIG. 5. If the user clicks on the title or picture of a newly listed lot, he will be sent first to a lot summary screen as shown in FIG. 7. FIG. 7 lists the pieces of equipment included in the lot, and allows the user to make an offer on the entire lot by clicking on “Make an Offer” button 18. Since the equipment in the lot is not being sold separately but rather together as a lot, the user makes an offer on the entire lot. From the screen shown in FIG. 7, the user can also put the lot on his watch list by clicking on “Watch this lot” button 21. Placing an item on a watch list saves the piece of equipment as a “favorite”, and allows the user to access it quickly in the future without digging through the categories to find it again. It should be understood that by clicking on the equipment picture or title in FIG. 7, the user can obtain more detailed information about a particular piece of equipment in the lot, in a similar manner as is done in FIG. 5.

The main screen shown in FIG. 2, and indeed the other screens, include a set of navigation buttons that when clicked on, take the user to other areas of the trading platform. The “Messages” button 22 allows the user to direct messages to individual members or groups of members, or direct messages to members having a certain role.

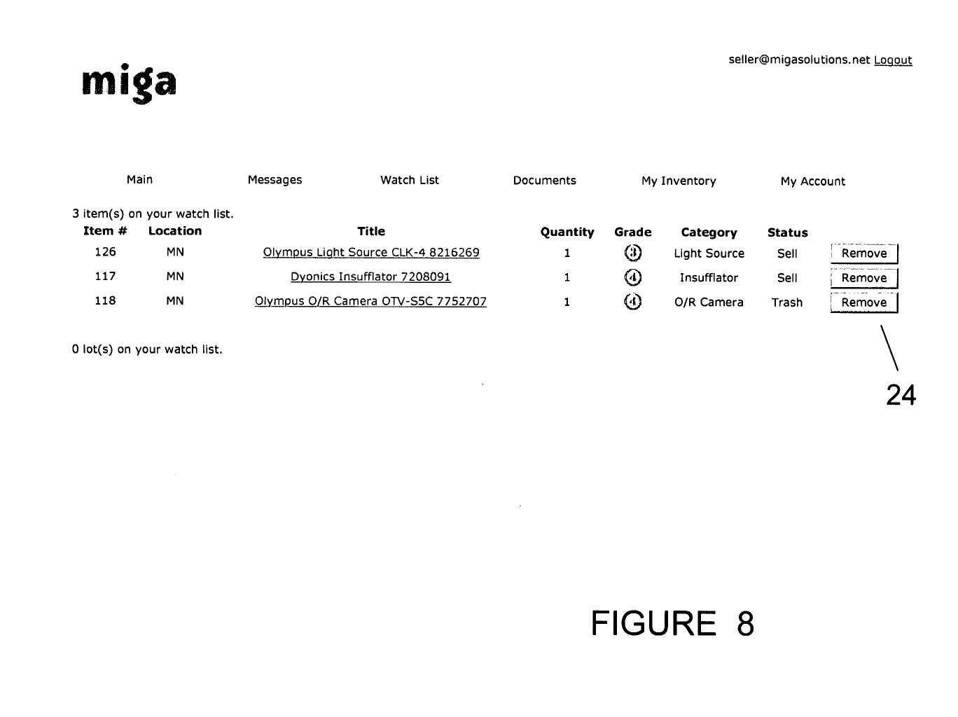

The “Watch List” button 23 takes the user to the screen shown in FIG. 8, wherein a list of the items the user has previously placed on his watch list is shown—both for individual items and multi-item lots. Information given in FIG. 8 includes the asset title, item number, location, quantity, grade (a condition indication), category/sub-category, and status (sell, trash, donate, etc.). The user can click on the item title to be taken to a primary information screen similar to that shown in FIG. 5—although this primary information screen will of course show detailed information corresponding to the requested piece of equipment, and not the specific piece of equipment shown in FIG. 5. Similarly, the user can click on the lot title to be taken to a lot summary screen similar to that shown in FIG. 7—although this lot summary screen will of course show detailed information corresponding to the requested lot, and not the specific lot shown in FIG. 7. Via the screen shown in FIG. 7, the user can also remove items from his watch list, by clicking on “Remove” button 24.

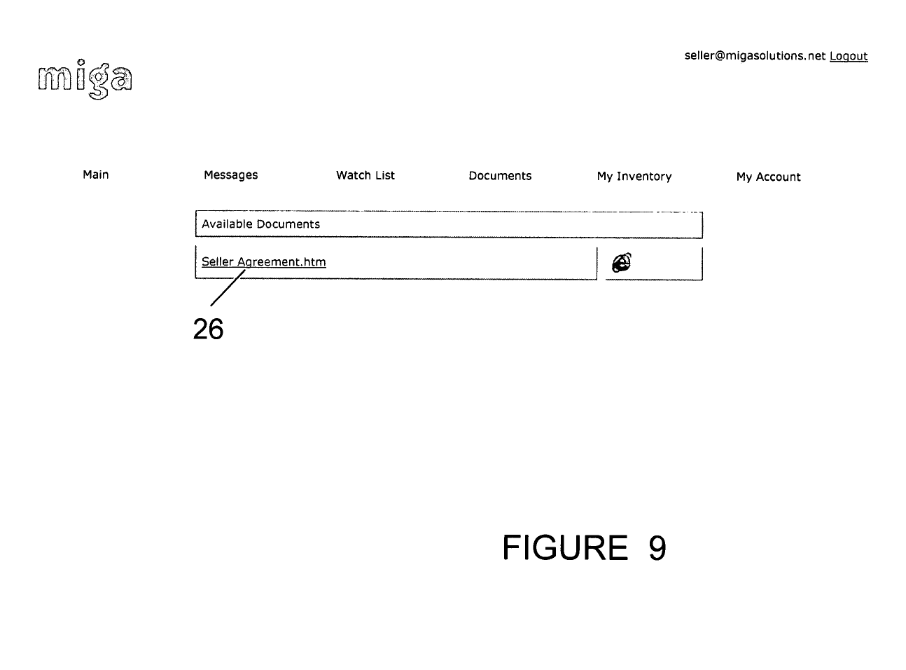

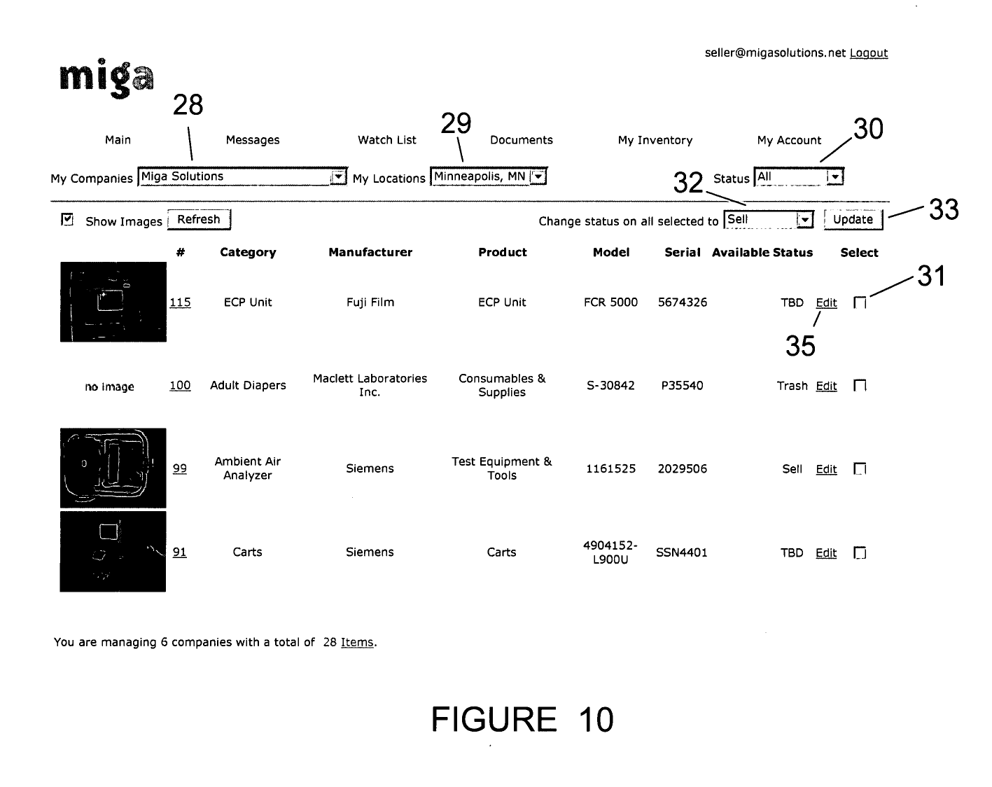

The “Documents” button 25 takes the user to the screen shown in FIG. 9, wherein the user can access various documents related to his account, including but not limited to seller's agreements, bills of sale, invoices, certificates of agency, etc. The user accesses such documents by clicking on the document title, such as the “Seller Agreement” button 26.

The “My Inventory” button 27 takes the user to the screen shown in FIG. 10. FIG. 10 shows the primary screen a user employs to view his equipment inventoried in the system, edit the information stored in the system about a particular piece equipment, and change the status of a particular piece of equipment—say, from “Sell” to “Donate”. Upon entering this screen, the user uses company drop-down menu 28 to select which of his companies he wishes to view the inventory for. The system allows a broker-user to represent many different companies, each of which can have separate inventories. For example, a broker might represent Mercy Hospital, Burnett Medical Center, and Hennepin County Medical Center, and be authorized to sell or otherwise dispose of equipment located at each of those hospitals. Since each hospital or asset-owning entity can have multiple locations, the user can also select the desired location via location drop-down menu 29. Finally, via status drop-down menu 30, the user can choose to view only those items having certain statuses, or view all the items by selecting “All” in status drop-down menu 30.

Once the user has defined his viewing parameters as discussed above, a list of items having those parameters is shown. The information given about each item includes the item number, a picture if available, category/subcategory, manufacturer, product description, model name/number, serial number, and status. Users can change the status of any item or set of items by selecting the items via select check box 31, selecting the desired new status via change status drop-down menu 32, and clicking on “Update” button 33.

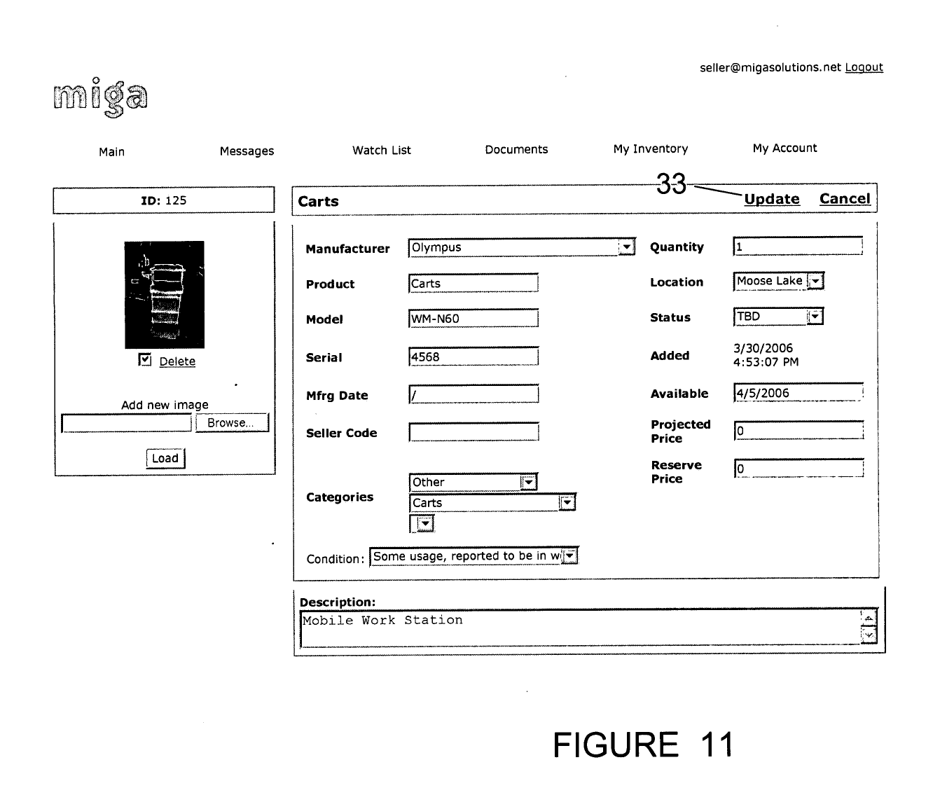

The user can also click on any item number via item number button 34, to be taken to a primary information screen similar to that shown in FIG. 5—although this primary information screen will of course show detailed information corresponding to the requested piece of equipment, and not the specific piece of equipment shown in FIG. 5. Further, the user can edit the information in the system for his items by clicking on “Edit” button 35. The user will then be taken to the screen shown in FIG. 11, wherein he can change the information for a particular item, delete the current picture of the item, or add a new picture. The user then clicks on “Update” button 33 to save the new information to the system.

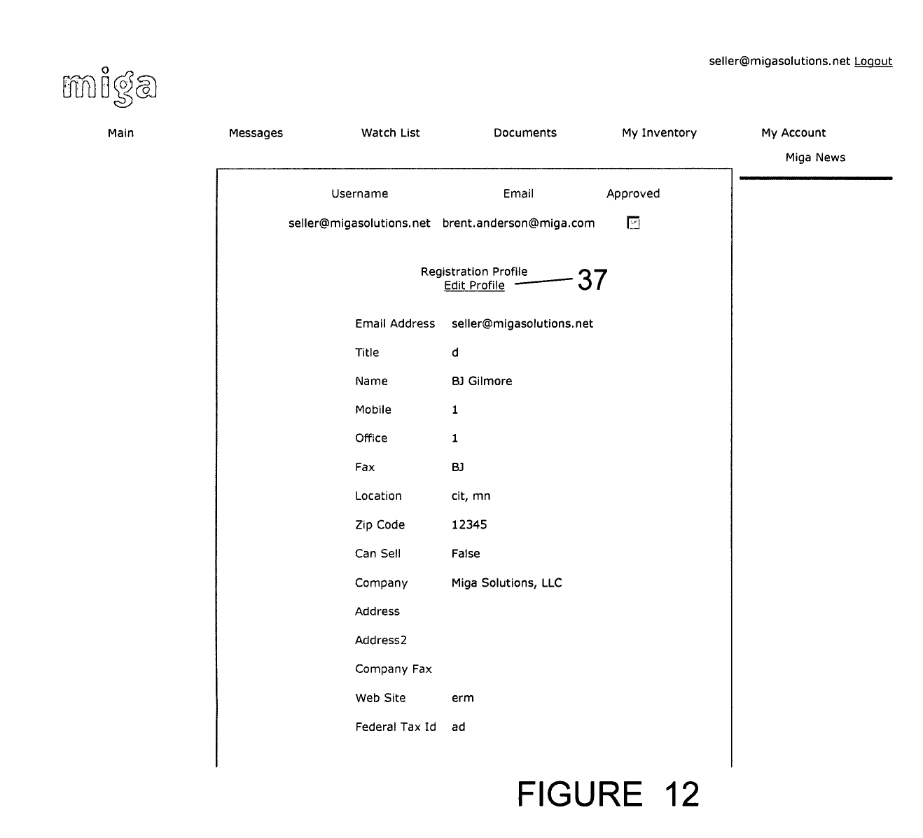

The “My Account” button 36 takes the user to the screen shown in FIGS. 12 & 13. This screen displays various information about the user, which makes up the user's registration profile. The user can edit his profile information by clicking on “Edit Profile” button 37. Finally, this screen also indicates whether or not the system administrator has approved or not approved the user for trading, via “Approved” button 38.

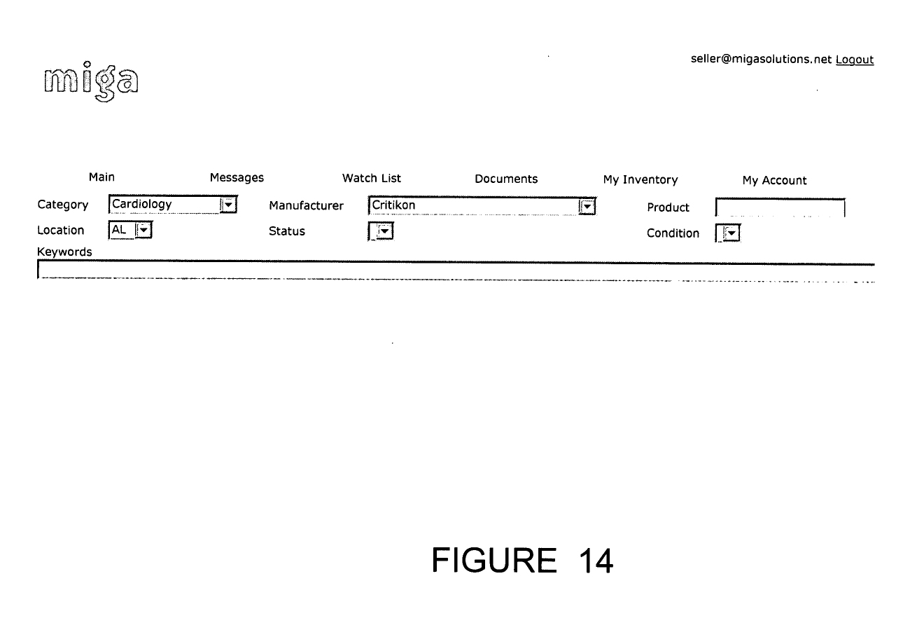

Via the screen shown in FIG. 2, the user can also search for particular pieces of equipment, using either quick search or advanced search facilities. To do a quick search, the user enters the search term into search text box 39 and clicks on the “Search” button to start the search. A list of the equipment associated with the search term is displayed. The user can click on a particular piece of equipment to access further information, make an offer for the equipment, or perform other tasks as have been described earlier. To do an advanced search, the user clicks on “Advanced Search” button 40, and is taken to the screen shown in FIG. 14. By selecting from drop-down menus and entering information into text boxes, the user can search for equipment by category, manufacturer, product description, location, status, or condition, and can also supply a keyword to further refine his search.

Several additional features of the trading module of the invention are described below:

### Wish List

The inventive system has a wish list feature, wherein a user identifies a piece of equipment that a user would like to obtain either by purchase or other means, and submits a request to the system to find that item. The wish list can contain two different types of items: 1) An item in the existing equipment inventory of the system, which the user does not want to purchase now but does want to purchase at a later date; or 2) An item that is not in the existing equipment inventory. For the latter type of item, the user enters various identifying information about the item, such as manufacturer, product, model, date needed, budget (the amount the user wants to pay), whether the user need that exact item or would be satisfied with a similar item, and other suitable information.

The user is notified when the item they are looking for is found by the system, and the notification can be via an email message or a notation on the user's account (in “My Account”). Once it is found and available, the wishlist item is placed in an online “shopping cart” for checkout and purchase. Wishlist items can be fulfilled either by the system or by other users or entities working together with the system in a network to find desired items. One example of a network approach that can be used is where the system builds a list of potential buyers and sellers, with potential sellers being told that a potential buyer is looking for and interested in buying a particular item. The wishlist can be saved by the user, such that the user can access it in the future, add to it, and subtract from it. A wishlist can be associated with a particular user, a group of users, or other entity (e.g., a certain department at a hospital composed of multiple users can share a wishlist).

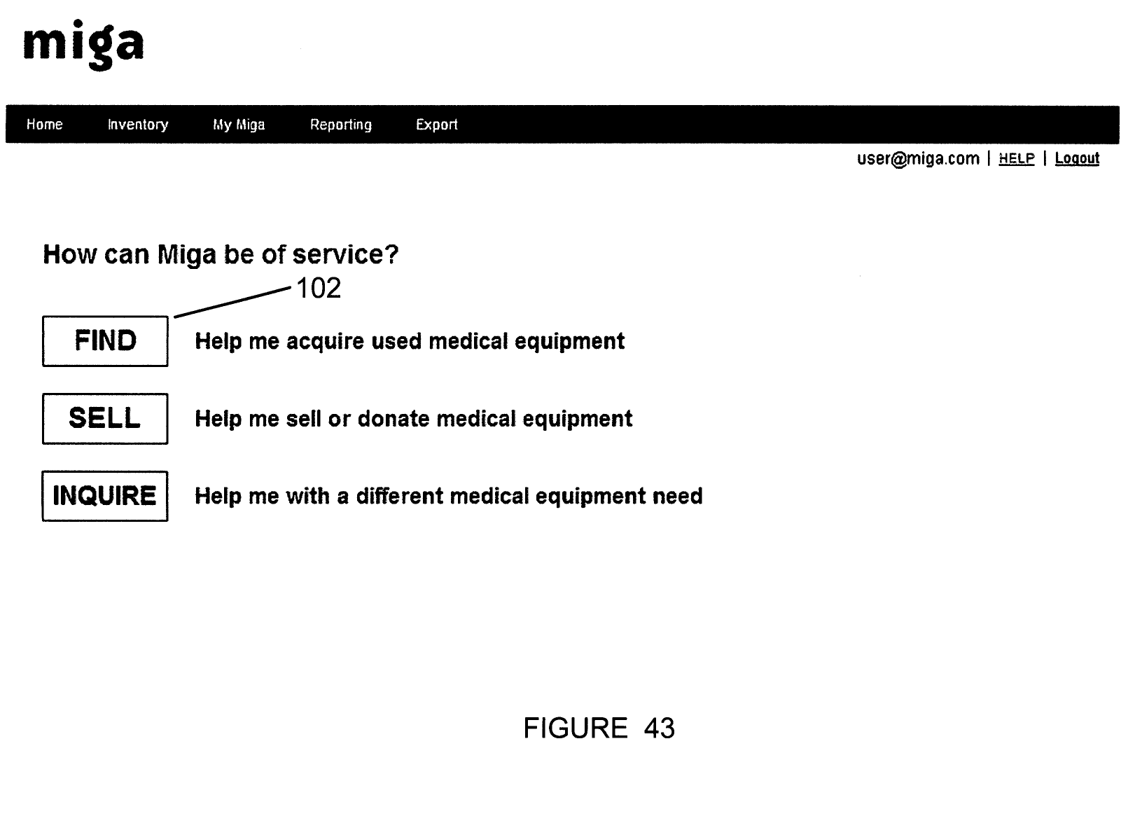

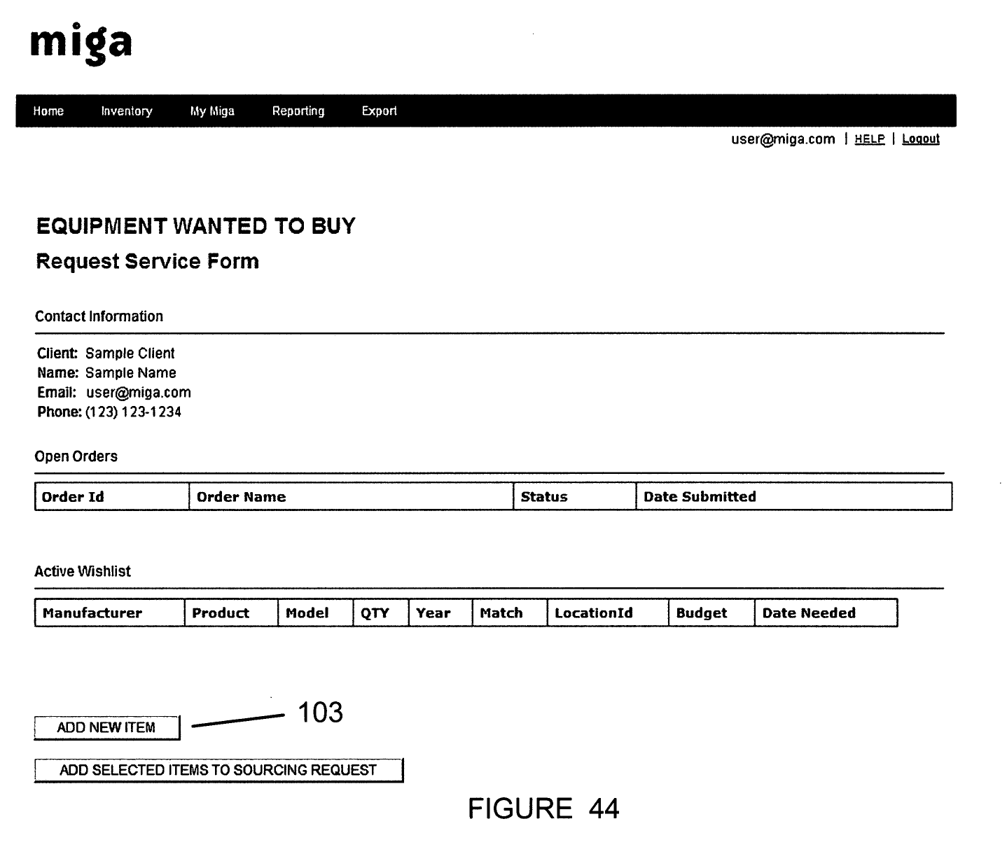

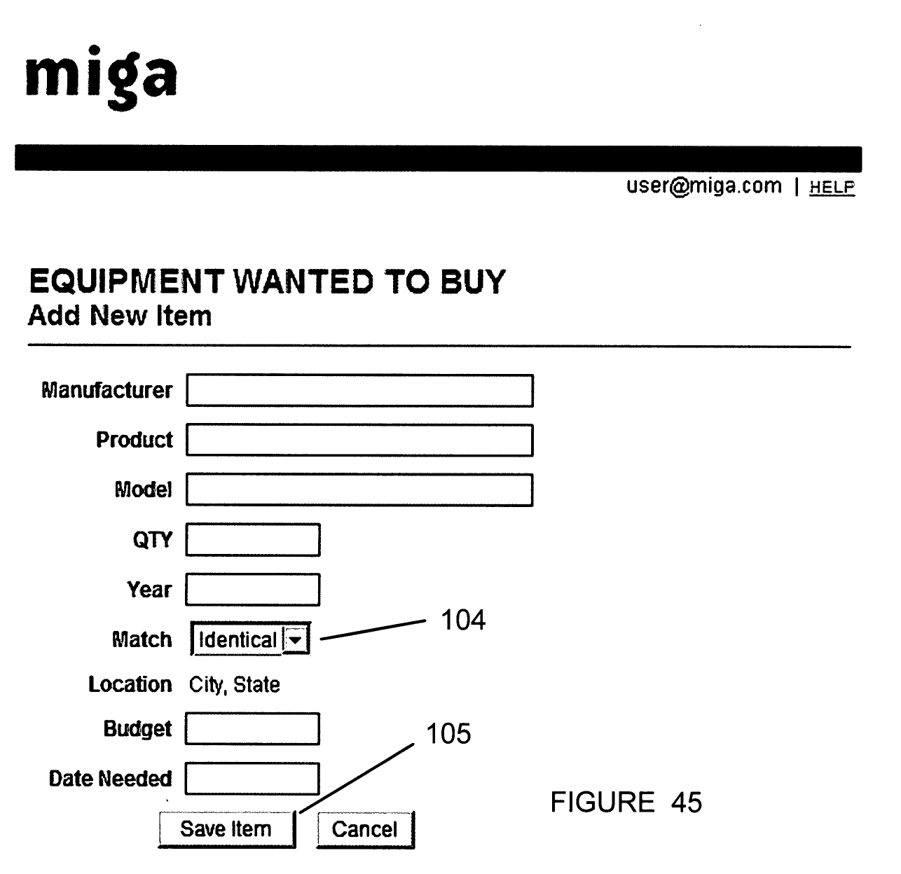

Operation of the wish list feature is further illustrated in FIGS. 43-48. After logging into the system, the user is taken to the website screen shown in FIG. 43. The user clicks on “Find” button 102 and is taken to the screen shown in FIG. 44 to compose a wish list of equipment the user wants to buy. The user clicks on “Add new item” button 103, and is taken to the screen shown in FIG. 45. The user adds a new item to his wish list by entering the appropriate data about the desired item—manufacturer, product, model, quantity (QTY), year of manufacture, his location (city and state), his budget for the item (how much he is willing to spend), and the date the item is needed. The user also clicks on match drop-down menu 104, and selects from “Identical” or “Similar” to indicate whether he needs an identical match or will accept a piece of equipment that is similar.

After the user has entered the data about his desired item, the data is checked for accuracy by the Gateway Module, using the same techniques discussed in the Gateway Module section of this application and shown in the associated figures.

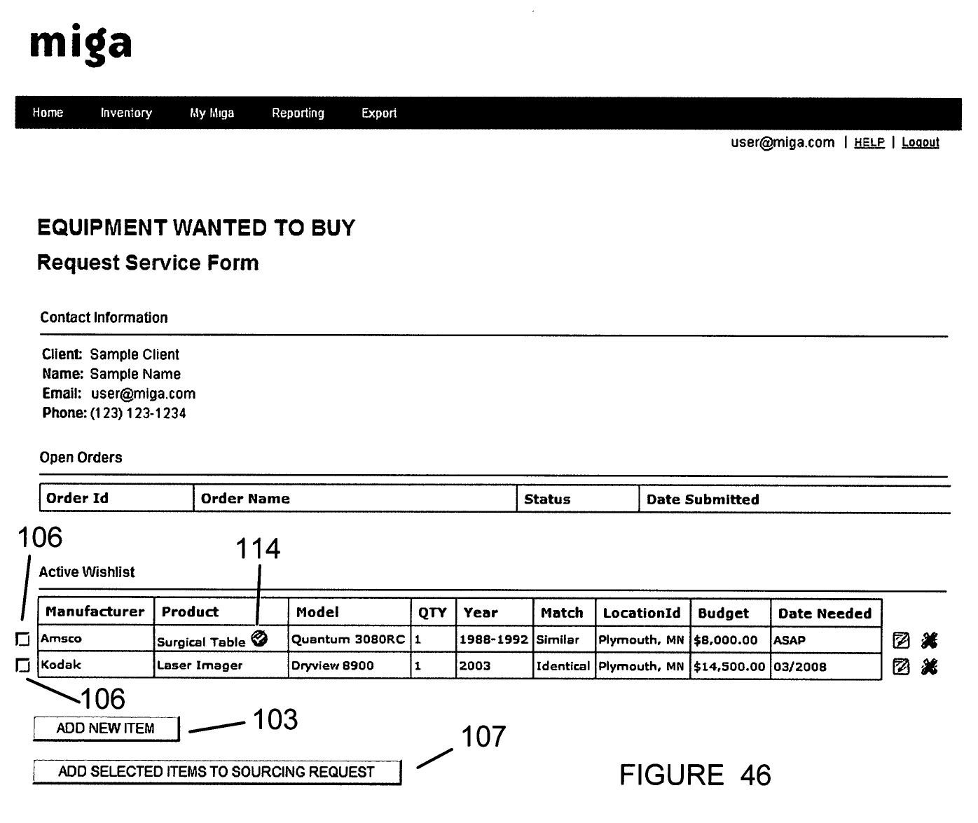

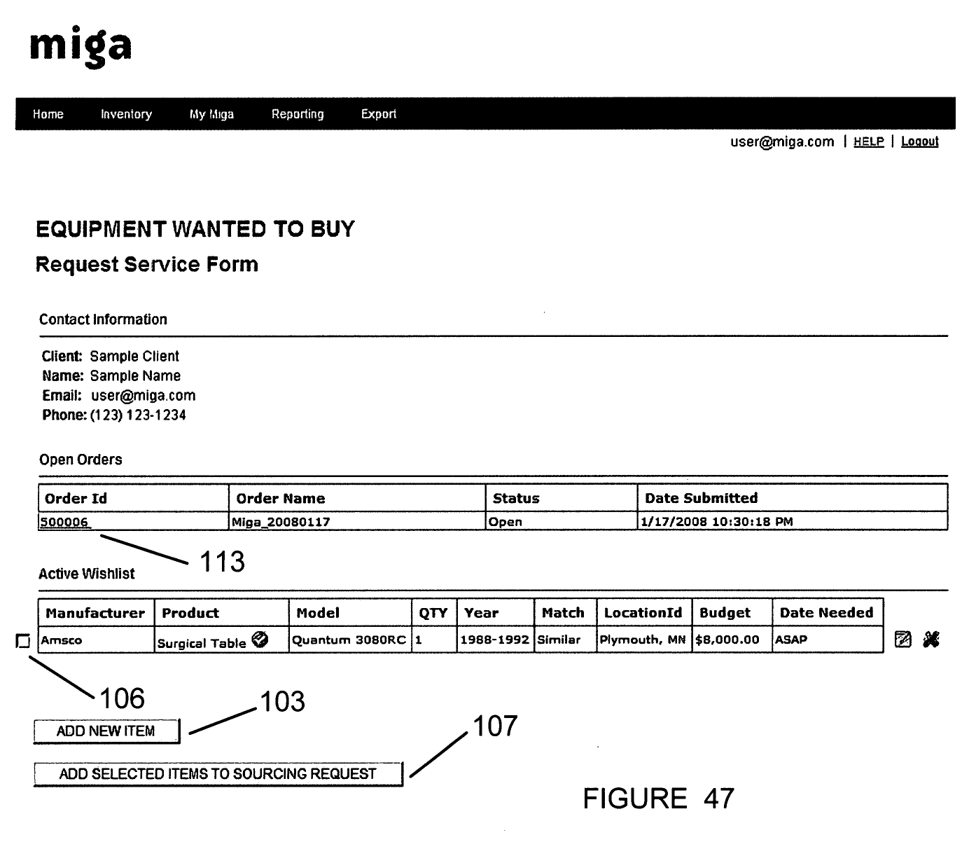

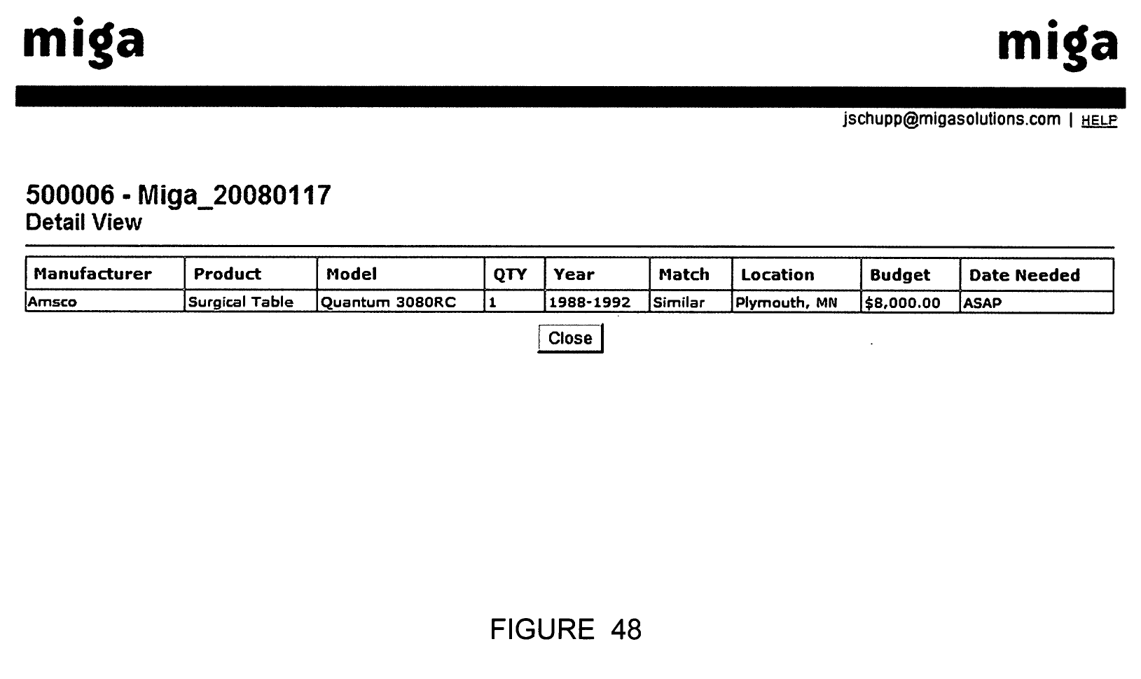

The user then clicks on “Save item” button 105, and the system saves the item to the user's wish list. The user is then taken to the screen shown in FIG. 46, wherein a list of the items in the user's active wishlist is displayed. It can be appreciated that the screen shown in FIG. 46 is similar to the screen shown in FIG. 44, except that now, the user has two items on his wish list—the Amsco surgical table and the Kodak laser imager. A “Found item” indicator 114 indicates to the user that a given item on his wishlist has been found by the system. Other items can be added to the user's wish list, if desired, by again clicking on “add new item” button 103. Further, the user moves items from the active wishlist to an open order status—thereby requesting that the system try to find that item—by checking one or more checkboxes 106 and then clicking on “Add new items to sourcing request” button 107. The screen shown in FIG. 47 illustrates the situation wherein one item is left in the active wishlist, and another item has been moved up into open order status thereby making a sourcing request to the system to find that item. When the user clicks on “Order ID” field 113, he is taken to the screen shown in FIG. 48, wherein details of the item that has been moved into open order status are provided.

### Redistribution

The inventive system also includes a redistribution feature, which facilitates the transfer of equipment from one location to another within the same entity—i.e., an “internal” exchange. Redistribution of equipment from one location to another within the same entity has also been referred to as the “transfer” option elsewhere in this application. Customer relationship management (CRM), or other suitable method, can be used to group users from the same entity together in order to link them and facilitate an internal exchange. Items posted to the internal exchange for redistribution are available for a limited time period before they are released to the public market. After items are released to the public market they are still available for redistribution, but will be competing with the interest of public buyers.

Internal exchanges of equipment are facilitated by establishing a fair market value (FMV) for the particular equipment, so that the receiving and sending parts of an organization can know the value of what is being redistributed. In the system, it is not required that the full FMV be paid from one part of an organization to the other, although it certainly could be. Instead, the minimum “price” is the amount that the system is owed for facilitating the redistribution. This allows a “seller” (the sending party in the redistribution) to give a significant discount to the “buyer” (the receiving party in the redistribution) if desired. For example, if the FMV=$10,000, and the system's fee for facilitating the redistribution is 20%, the item may sell for $2,000 at a minimum (all proceeds go to the system), and anything additional up to $10,000 would go to the seller. Redistribution can be done for any type of equipment—medical, technology (computers, etc.), disposables, furniture, etc.

The online offer management features of the system facilitate the negotiation of FMV, purchase price, and other various logistics including arranging for the actual physical transfer/shipment of the redistributed item. Additional aspects of the redistribution functionality include reporting, physical transfer of the item (project management, logistics, handling, de-installation), and non-physical/electronic aspects of the item transfer (financial entries, changes to asset management systems and cost centers for both the receiving and sending parts of the organization). Further, the internal parties can use the system as a relatively apparent intermediary, or alternatively the system can employ software elements that “reach into” each internal party so that the role of the system in the redistribution is transparent to the internal parties. In this latter alternative, the user screens associated with the redistribution have the look and feel of the organization within which the redistribution is occurring, rather than having the look and feel of the system's screens.

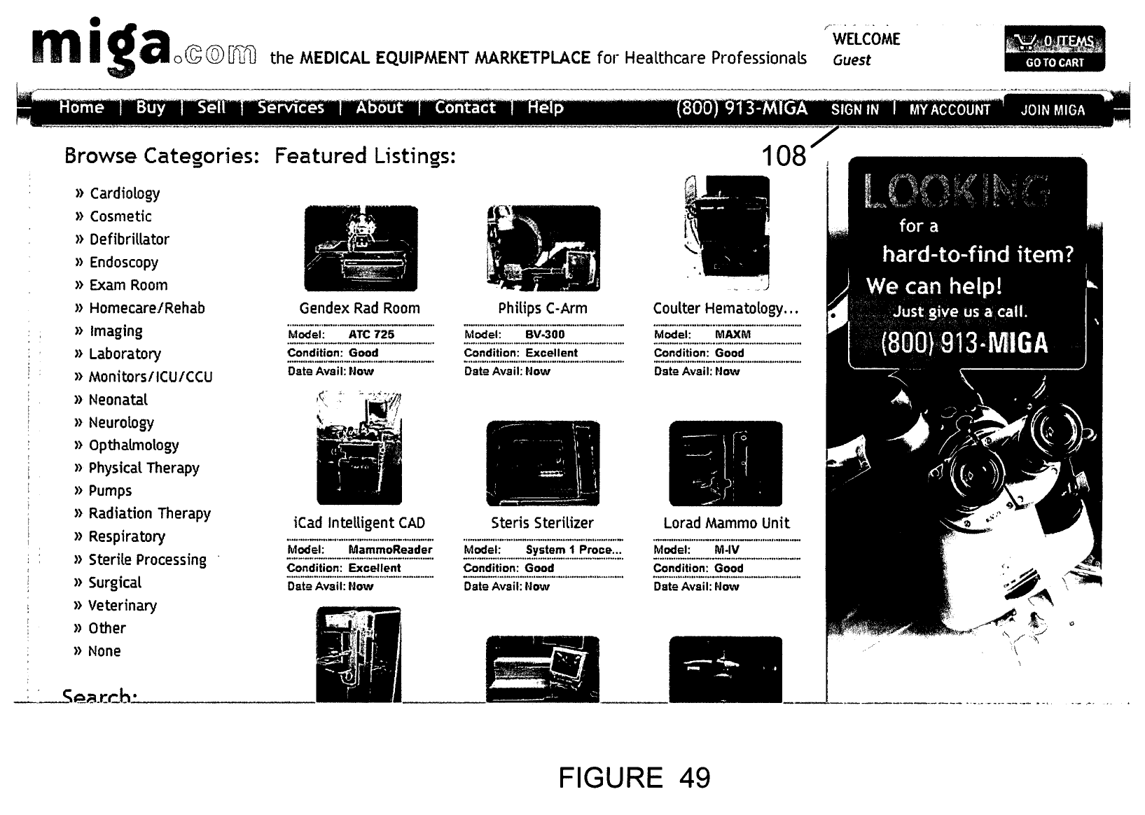

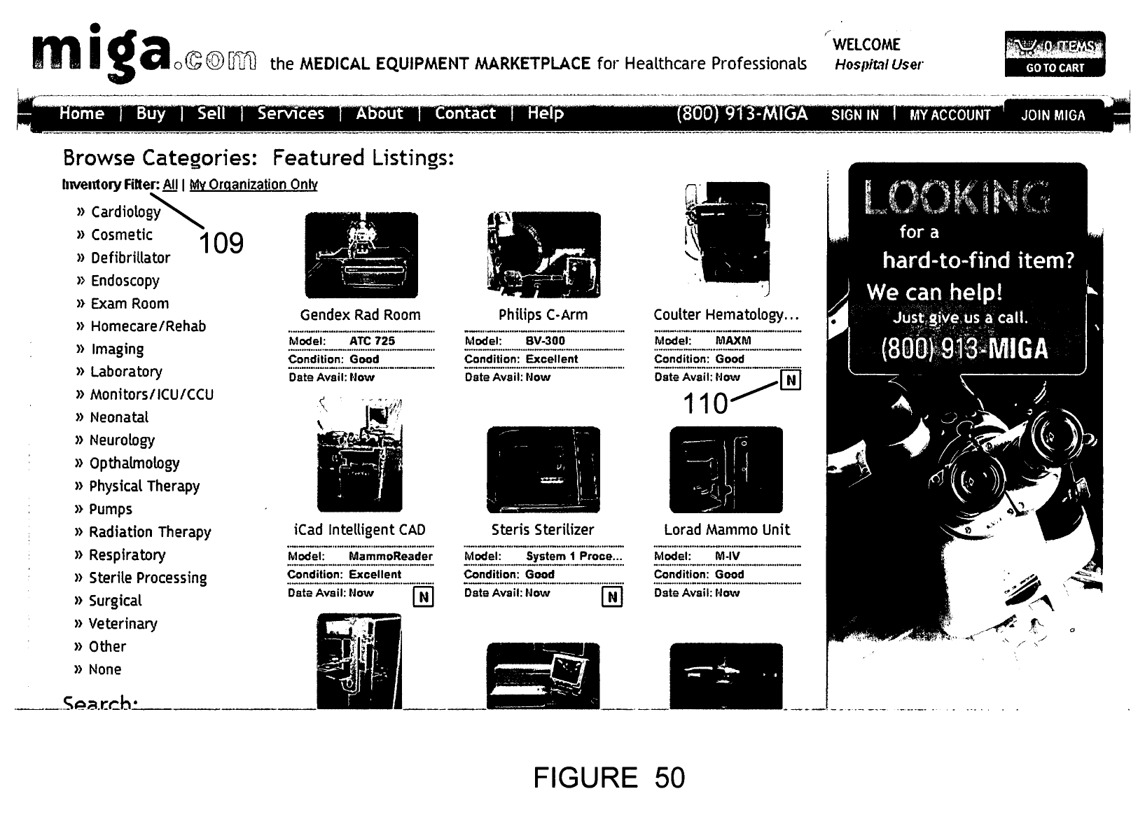

Operation of the redistribution feature is further illustrated in FIGS. 49-54. The screen shown in FIG. 49 illustrates an alternative embodiment of the trading platform home page. The user clicks on “Sign in” button 108, and logs into the trading platform. The user is taken to the screen shown in FIG. 50, wherein the user can browse the equipment available on the trading platform. It should be noted here that the equipment that is shown to the user, and what the user is allowed to do on the system, can depend on the user's profile (e.g., hospital user, guest, non-profit, etc.) and the inventory filter that is employed. The system knows that a particular user is part of a larger organization via the user ID the user enters during the log-in process—in other words, a particular user's log-on ID is pre-identified with a larger organization during the forming of the user's profile.

The inventory filter of the invention serves to filter the equipment shown to the user, through the use of various parameters including “All” (all equipment in the system is shown); “My organization only” (only the equipment offered by other members of the user's organization—say, other members of a hospital or health network—is shown); and other suitable parameters. The inventory filter options are operable by “Inventory filter” buttons 109—the user can toggle between showing all the equipment or just the equipment offered by members of the user's organization. It can be appreciated that in FIG. 50, all the equipment is shown—items marked with a “Item in my organization” indicator 110 (here, the letter N) as well as unmarked items which are offered by others who are not in the user's organization. Alternatively, the user can see just the available equipment in his own organization by clicking on the “My organization only” portion of “Inventory filter” button 109.

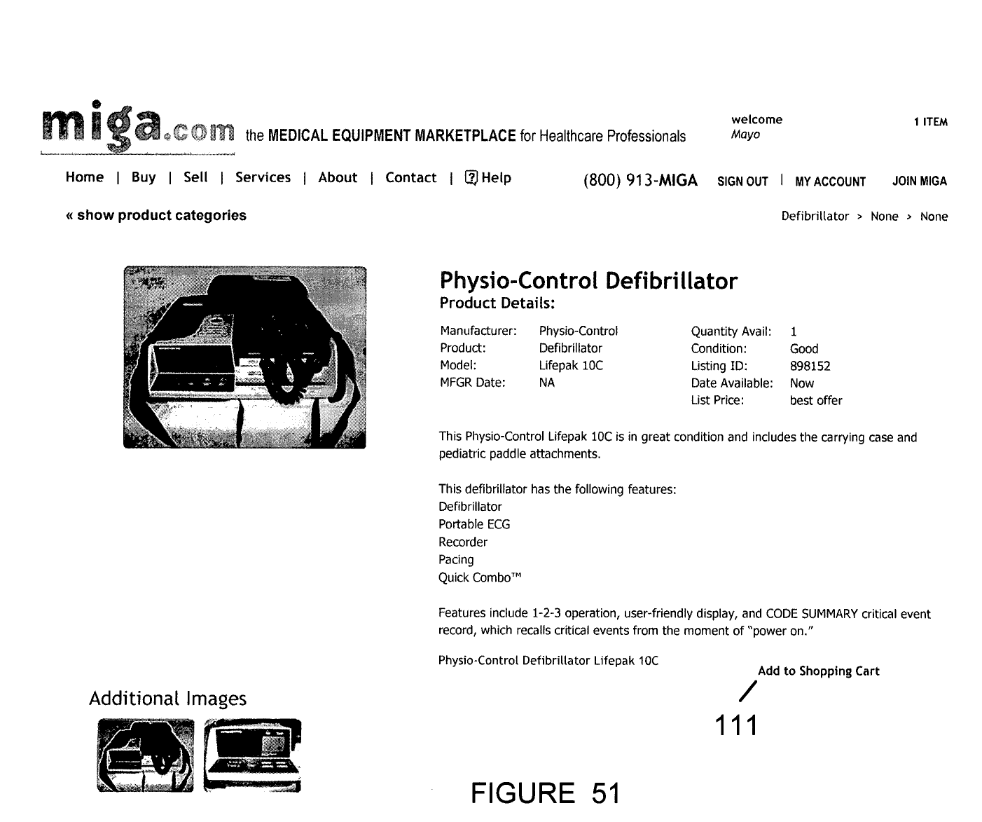

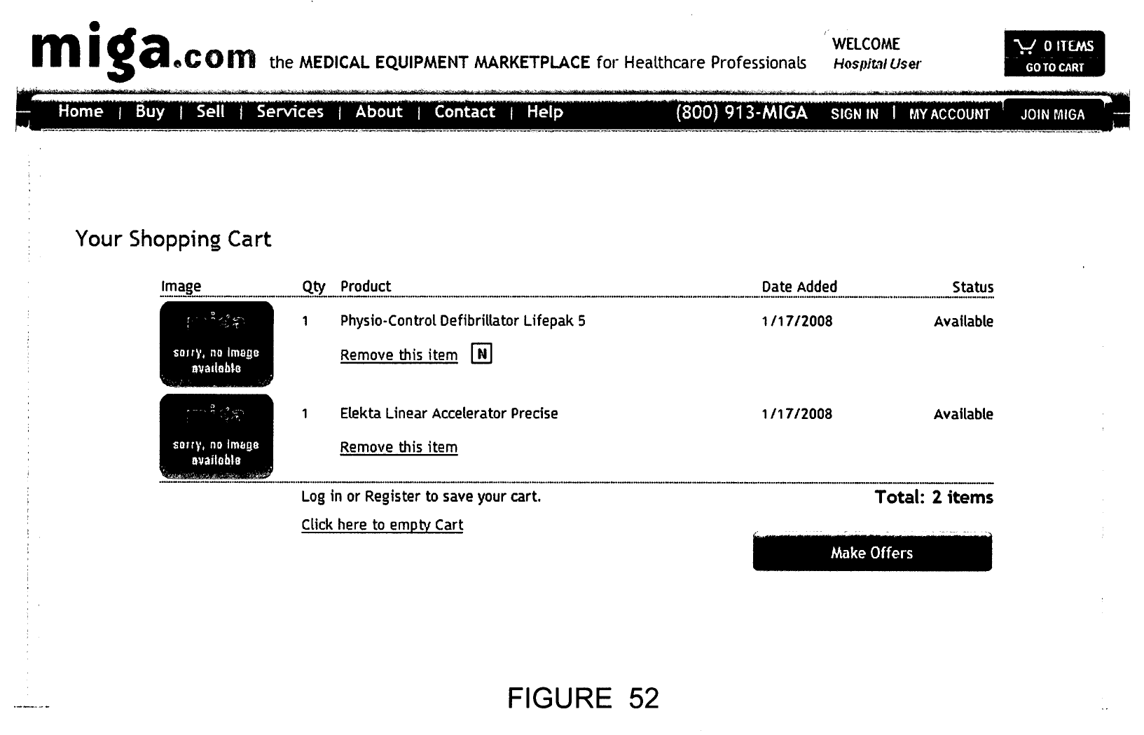

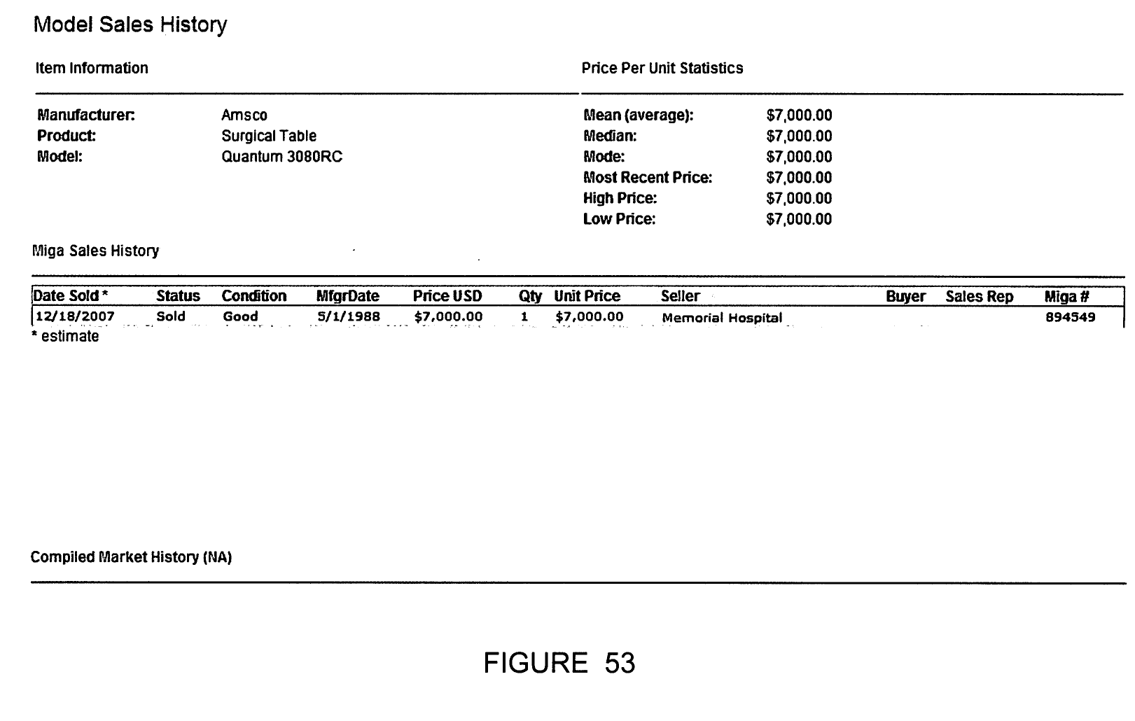

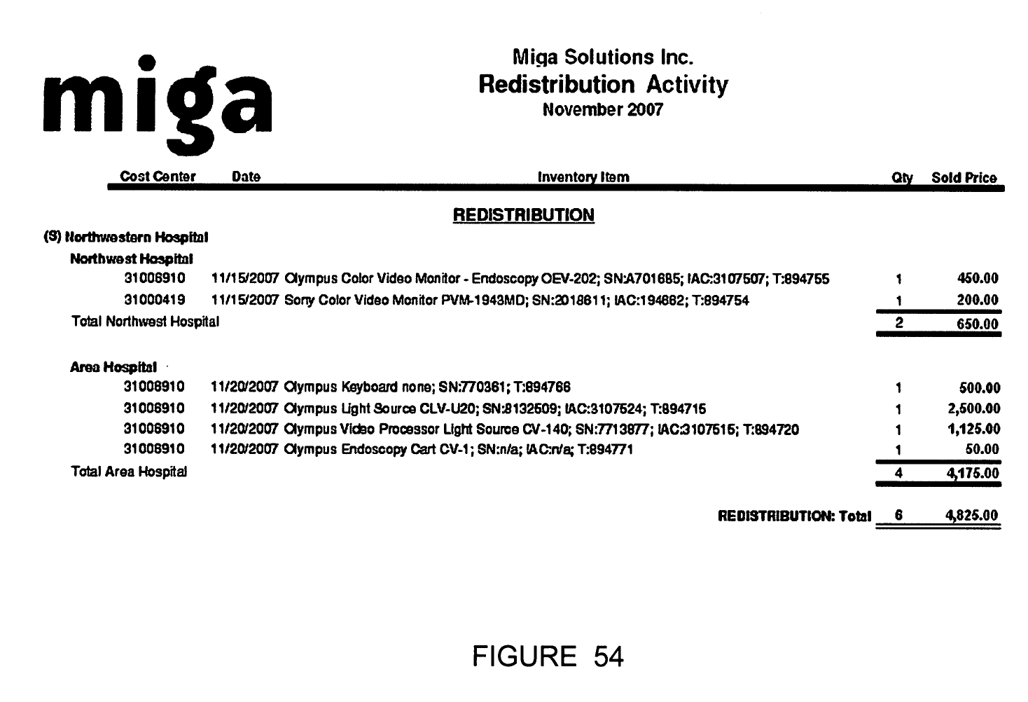

When the user sees an item of interest, he clicks on it and is taken to the screen shown in FIG. 51, which provides detailed information concerning that item. If still interested, the user clicks on “Add to shopping cart” button 111 to add the item to his shopping cart, and is taken to the screen shown in FIG. 52. FIG. 52 illustrates the user's shopping cart, and in this case there are two items of equipment in the cart—one item that is offered by a member of the user's own organization, and that represents a redistribution of equipment within the organization; and another item offered by another entity that is not in the user's organization. FIG. 53 shows a Model Sales History feature, wherein the user can see the sales history of a given item on the trading platform along with various other information on the item, such as condition, seller, date sold, etc. Further, the user is shown price statistics for the given item, such as the price mean, median, and mode; the most recent price, and the high and low prices at which the item changed hands. This information is used to establish the fair market value (FMV) for a particular piece of equipment; the FMV being the “price” at which the equipment is redistributed/transferred from one part of an organization to the other. FIG. 54 shows a Redistribution Activity report, wherein the user can see the redistribution activity for a given period—here, November 2007. Each piece of equipment that was redistributed during that time period is listed, along with various information about the equipment such as date sold, manufacturer, model name, serial number, etc.

### Donation

The inventive system also includes a donation feature, which creates an electronic exchange where non-profit entities can view and request goods that are offered for donation. Non-profit entities register with the system, and their qualifying status is verified by the system so that donors can be assured that their donation will be tax-deductible. The system may require information concerning the non-profit's 501(c)(3) certification by the federal government to prove or support an entity's qualifying status, as well as other proofs and certifications such as Federal ID# (IRS Form SS-4), a Charities Registration Certificate, the entity's Provisional Charter or Articles of Incorporation (for historical societies, museums, cultural agencies, educational organizations, public safety groups, scientific organizations, etc.), or an Exempt Organization Certificate. The system can also match certain preferred non-profit entities with existing donors, such that the non-profits preferred by a given donor get the “first look” at any assets donated by that donor—i.e., before the donated assets are broadly shown to all non-profits.

The system establishes the fair market value (FMV) of donated items. This facilitates the reporting of the donation transaction to external authorities such as taxing authorities, and also aids in the internal bookkeeping done by the donor and donee. The FMV can be established in several ways, by using: 1) A price history for the asset—i.e., what the asset sold for previously and when; 2) A network of buyers and sellers, either internal or external to the system, that are familiar with that type of asset and can accurately value it; and 3) Price information obtained by market research, which identifies what similar assets are selling for on eBay, Google, dot.med, and other marketplaces. The system also provides supplemental logistic and project management support to the donor and donee, as needed. For example, the system can help the donor and donee plan and manage the de-installation process for the asset, and arrange for shipping and handling. Further, the system allows non-profits to access, filter, identify, and browse items available for donation. The non-profit user or entity can also see and browse items that are offered for sale, not just items that are offered for donation. By using an inventory filter, the non-profit user can opt to see only donation items, only for-sale items, or a combination of donation and for-sale items wherein the donation items are marked to indicate their donation status. Often, non-profit entities also have a budget to purchase needed items and are more likely to purchase these items from a seller who is also donating items to them. Thus, permitting non-profits to see items for sale as well as items for donation, and “mix and match” among these items when making a transaction, is valuable. In addition, for-sale items can be sold to a non-profit at a discount off the listed price—in other words, a middle ground between straight sale and straight donation.

The system provides detailed tracking and reporting back to the donor of donation transactions. The system also provides integrated approval for the donation process (from donors) via contractual or electronic notification. That is, identification and approval of the items to be donated can be pre-arranged by contract, or be done via electronic notification. Specifically, in the contractual option, the system is pre-authorized to make items available for donation at its own discretion or based on various criteria such as previous item history, length of time the asset has remained unsold, etc. After the system has determined that certain items should be donated according to the contractual terms, it may optionally notify the seller, and then optionally the seller may have a set period of time to object to the donation before it is finalized. In the electronic notification option, after determining that a certain item should be donated the system asks for approval from the seller before the item is switched to donation status.

Further, the system can automatically transfer, electronically, items for donation to a charitable foundation while still displaying them within the system. Items so transferred can items be withdrawn from the charitable foundation with no obligation, and this is solely at the discretion of the foundation. In facilitating the donation, the system can act as a relatively apparent intermediary, or alternatively the system can employ software elements that “reach into” the donor and donee so that the role of the system in the donation is transparent to them. In this latter alternative, the user screens associated with the donation have the look and feel of the donor or donee organization, rather than having the look and feel of the system's screens.

The wishlist feature described herein also operates with donated items—i.e., a user may place both donated and for-sale items on his wishlist. In addition, it is not unusual for a for-profit entity to be interested in a hard-to-find item that is only available as a donation item. Accordingly, the system can also display donated items to for-profit entities, and for-profit entities can obtain such items. However, the handling is different—if a donated item is obtained by a for-profit entity, the proceeds of the transaction (for example, the FMV if the for-profit entity pays FMV) go to a charitable foundation—either a foundation external to the system, or one set up and managed by the system.

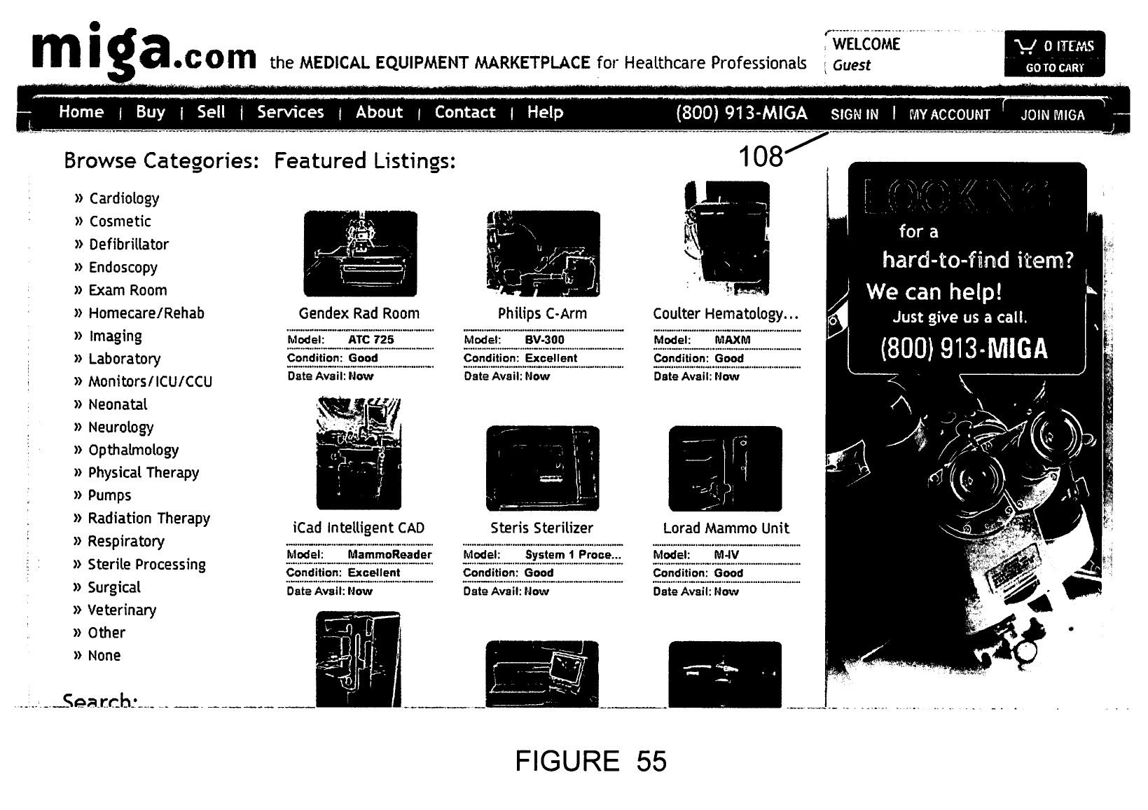

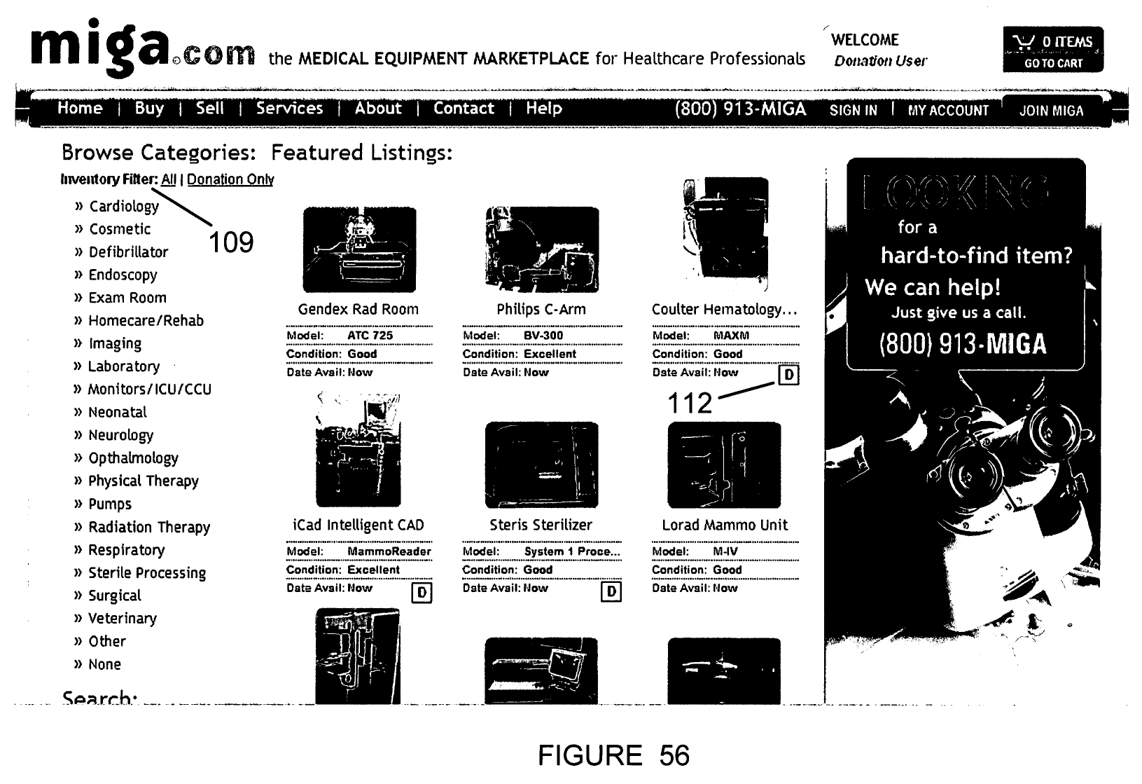

Operation of the donation feature is further illustrated in FIGS. 55-59. The screen shown in FIG. 55 illustrates an alternative embodiment of the trading platform home page. The user clicks on “Sign in” button 108, and logs into the trading platform. The user is taken to the screen shown in FIG. 56, wherein the user can browse the equipment available on the trading platform. It should be noted here that the equipment that is shown to the user, and what the user is allowed to do on the system, can depend on the user's profile (e.g., for-profit or non-profit, etc.) and the inventory filter that is employed. The system knows that a particular user is part of a qualifying non-profit organization (e.g., a 501(c)(3) organization) via the user ID the user enters during the log-in process. In other words, a particular user's log-on ID is pre-identified with a qualifying non-profit organization during the forming of the user's profile. Donations to qualifying non-profit organizations are tax-deductible to the donor, and thus if the user is part of such an organization, the system allows him access to items offered for donation.

The inventory filter of the invention serves to filter the equipment shown to the user, through the use of various parameters including “All” (all equipment in the system is shown); “Donation only” (only the equipment offered for donation is shown); and other suitable parameters. The inventory filter options are operable by “Inventory filter” buttons 109—the user can toggle between showing all the equipment or just the equipment offered for donation. It can be appreciated that in FIG. 56, all the equipment is shown—items marked with a “Donation item” indicator 112 (here, the letter D) as well as unmarked items which are offered for sale or other forms of disposition, but not for donation. Alternatively, the user can see just the equipment available for donation by clicking on the “Donation only” portion of “Inventory filter” button 109.

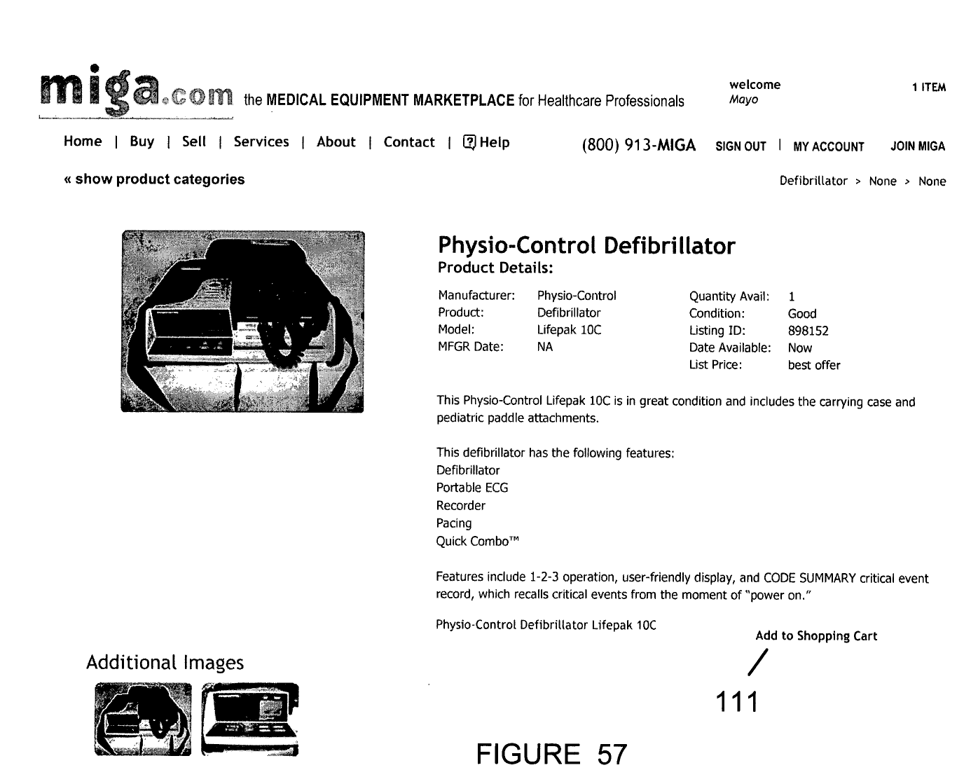

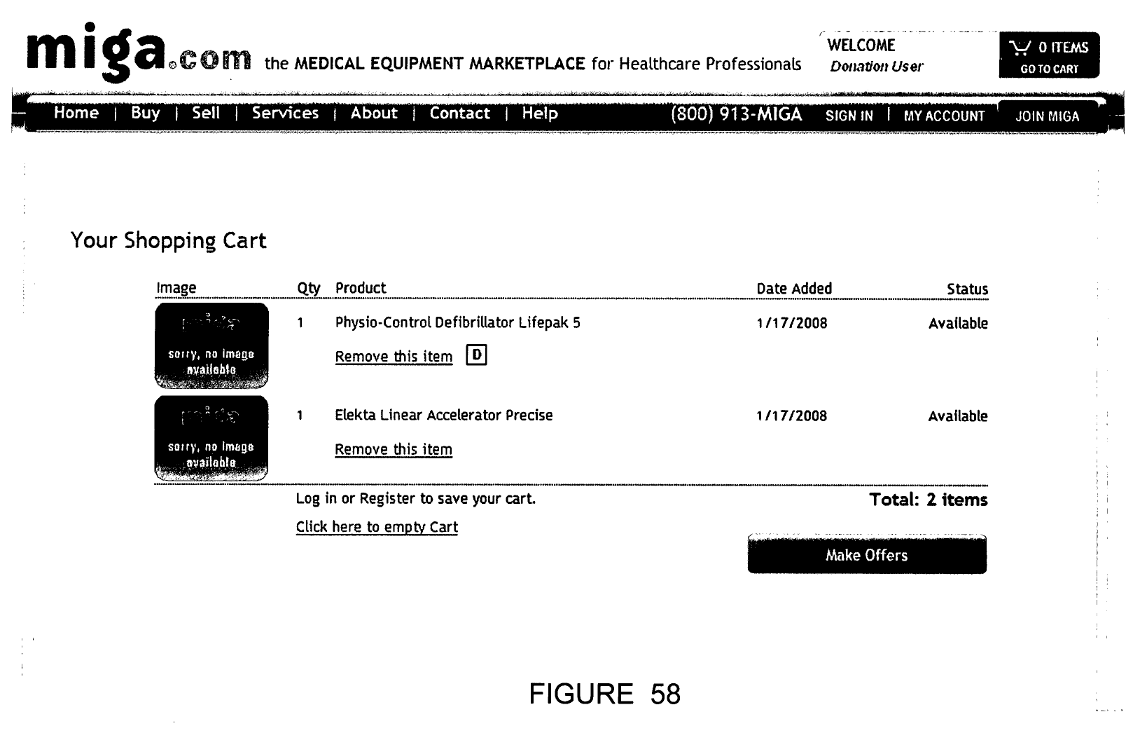

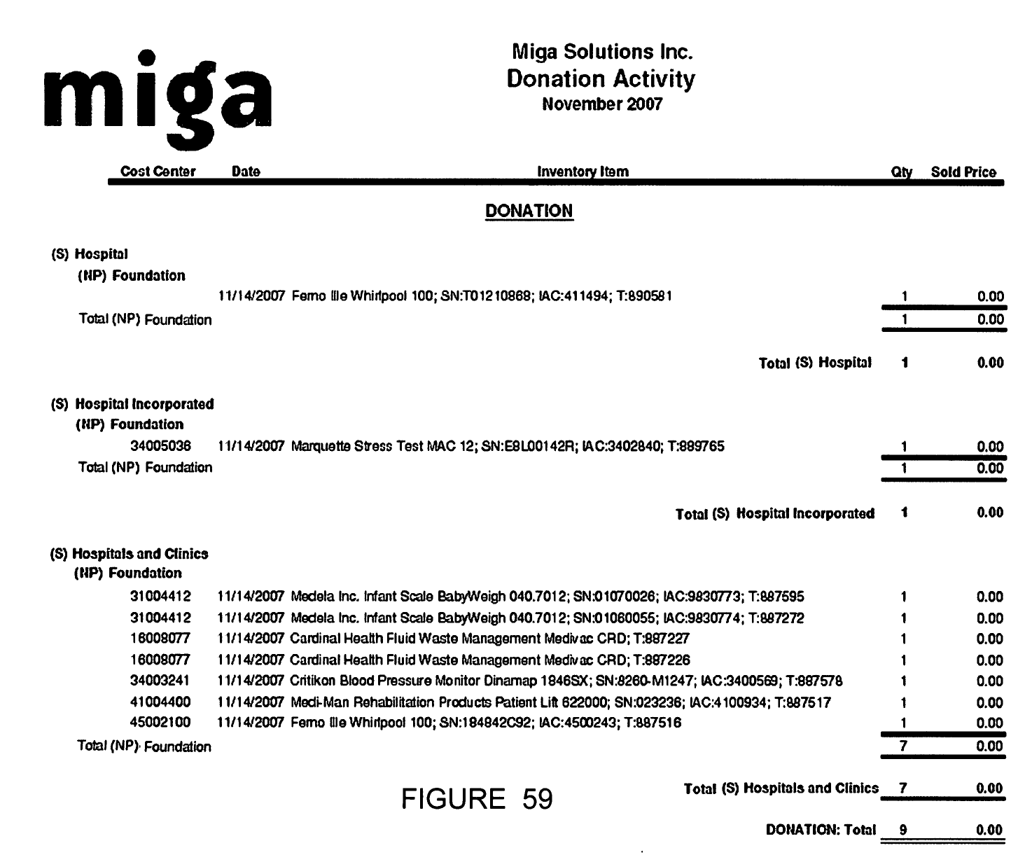

When the user sees an item of interest, he clicks on it and is taken to the screen shown in FIG. 57, which provides detailed information concerning that item. If still interested, the user clicks on “Add to shopping cart” button 111 to add the item to his shopping cart, and is taken to the screen shown in FIG. 58. FIG. 58 illustrates the user's shopping cart, and in this case there are two items of equipment in the cart—one item that is offered for donation; and another item offered for sale. It can be appreciated that a qualifying non-profit user can accept a donated item as well as buy an item offered for sale. FIG. 59 shows a Donation Activity report, wherein the user can see the donation activity for a given period—here, November 2007. Each piece of equipment that was donated during that time period is listed, along with various information about the equipment such as date sold, manufacturer, model name, serial number, etc.

It should also be understood that the information in FIG. 53 is also used in the donation process, in order to establish the fair market value (FMV) for a particular piece of donated equipment. The FMV is used by the donor when requesting the tax deduction associated with the donation.

### Administrative Realm

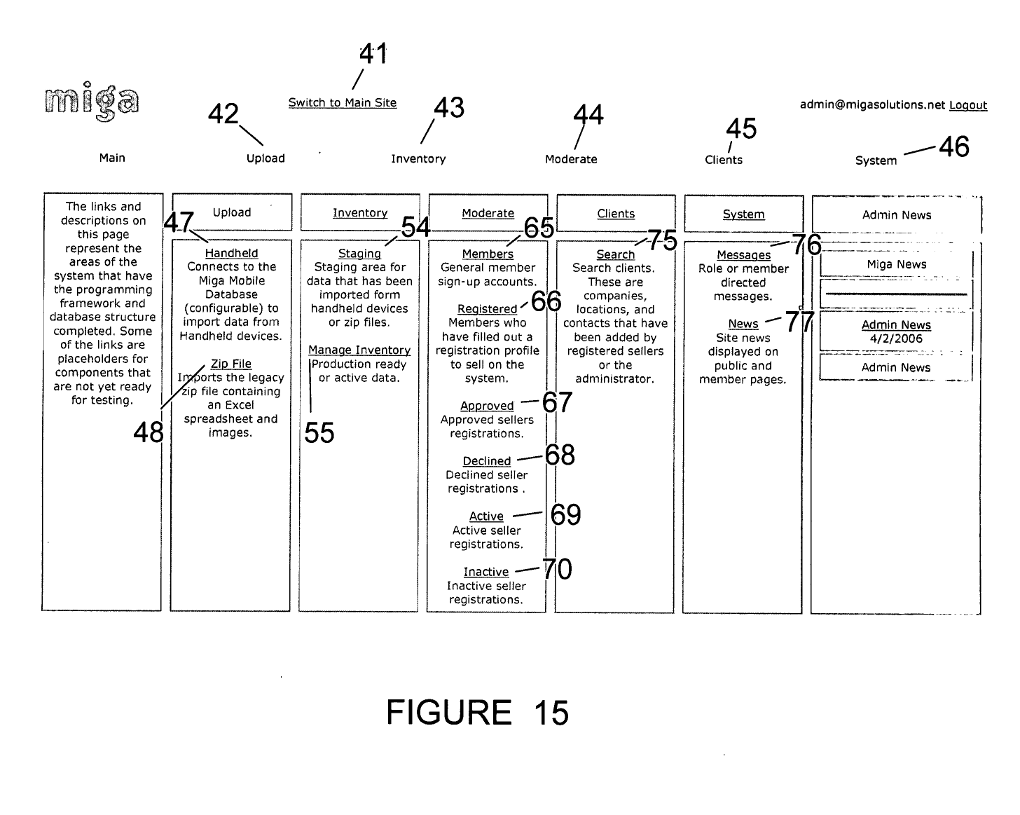

In addition to the user realm, the trading platform also has an administrative realm, comprised of screens the system administrator uses to administer and maintain the system. The administrative realm is reached by logging onto the trading platform using a username and password that has administrative privileges. Once logged on, the administrator is taken to a main screen shown in FIG. 2; however, this screen has a “Switch to Administrative Site” button that the administrator clicks on to be taken to the main screen of the administrative site, as shown in FIG. 15. FIG. 15 has a “Switch to Main Site” button 41, so that the administrator can easily toggle back and forth between the user site described earlier and the administrative site.

The main administrative page shown in FIG. 15 has a number of navigation buttons arrayed across the top of the page, including “Upload” button 42, “Inventory” button 43, “Moderate” button 44, “Clients” button 45, and “System” button 46. When the administrator's cursor is passed over each of these navigation buttons, a menu of sub-selections is revealed. The administrator clicks on his desired sub-selection, and is taken to an appropriate page to manage that sub-selection. The sub-selections are also listed below each main navigation button, so that the administrator may click directly on a particular administrative function to access the screen or screens associated with managing that function. The main navigation buttons, sub-selections, and administrative functions associated with the sub-selections are further described below:

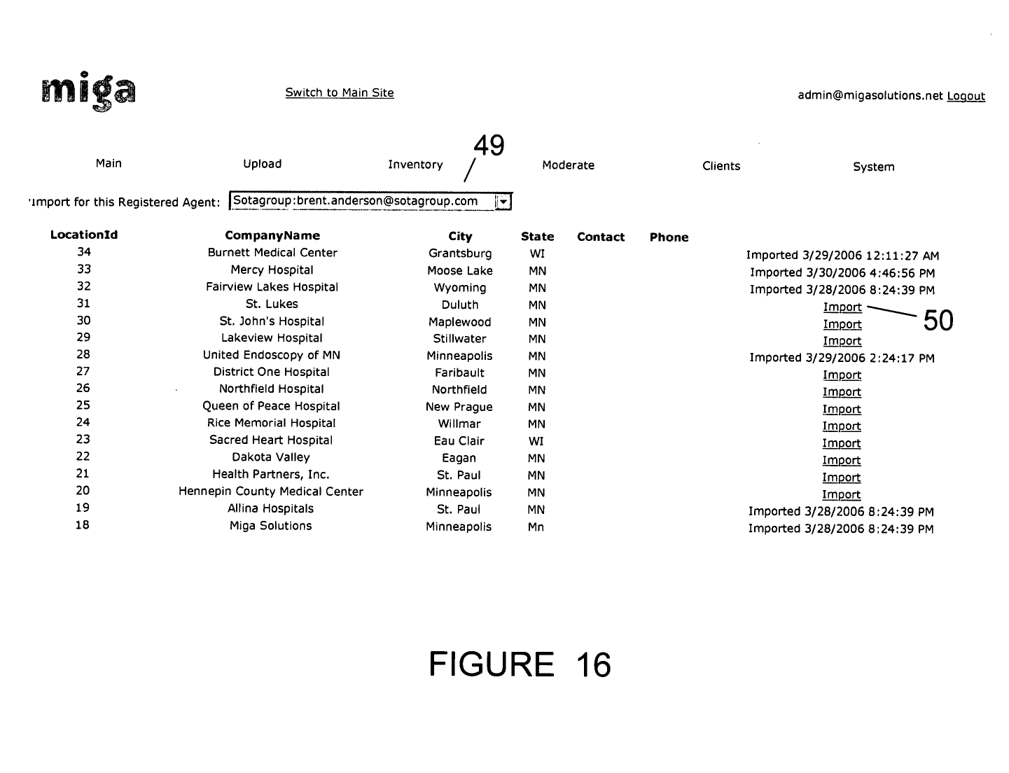

“Upload” button 42 and its two associated sub-selections, reached by moving the cursor over “Upload” button 42 and making a selection, or via “Handheld” button 47 and “Zip File” button 48, allow the administrator to import equipment data into the trading platform. When the administrator clicks on “Handheld” button 47, he is taken to the screen shown in FIG. 16, and connects to the equipment database. The administrator can import data into a staging area (also referred to as a “staging table”) of the system—specifically, data that was originally input into the equipment database by data input modules such as the handheld devices described earlier. On the FIG. 16 screen, the administrator first selects the registered agent (registered agents can manage assets for a number of different entities or companies—in this case hospitals) via “Registered Agent” drop-down menu 49. A list of all the equipment managed by that agent—both previously-imported into the trading platform and not-yet imported—is displayed.

Information about each piece of equipment is shown, including its location ID, the name of the company owning it and the company's contact information, and whether the equipment has been imported into the trading platform or has yet to be imported. If the equipment has been imported into the trading platform, the date and time it was imported are shown. If the equipment has yet to be imported, an “Import” button 50 is displayed. To import the equipment into the staging area/staging table, the administrator clicks on “Import” button 50.

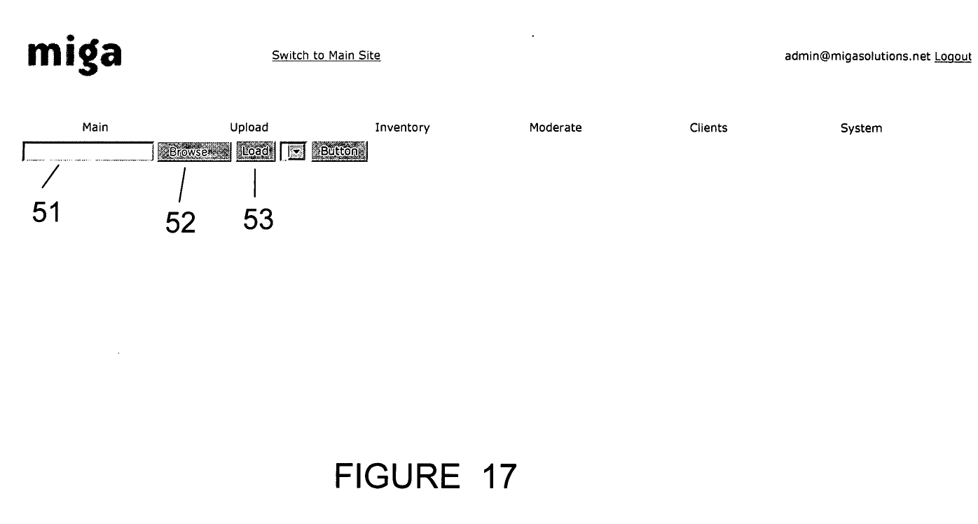

The administrator can alternatively load equipment into the staging area by uploading previously prepared files, including but not limited to Zip files. When the administrator clicks on “Zip File” button 48, he is taken to the screen shown in FIG. 17. The administrator either enters the file pathname into file text box 51, or clicks on “Browse” button 52 to locate the desired file. Once the desired file has been entered or located, the administrator clicks on “Load” button 53 to upload the equipment data into the staging area.

Returning now to FIG. 15, “Inventory” button 43 has two associated sub-selections, reached by moving the cursor over “Inventory” button 43 and making a selection, or via “Staging” button 54 and “Manage Inventory” button 55. “Staging” button 54 allows the administrator to view and modify data about equipment in the staging area that has been imported from handheld devices or from files. “Manage Inventory” button 55 allows the administrator to view and modify data about equipment that is active in the trading platform—i.e., equipment that is available for sale and accessible by trading platform users.

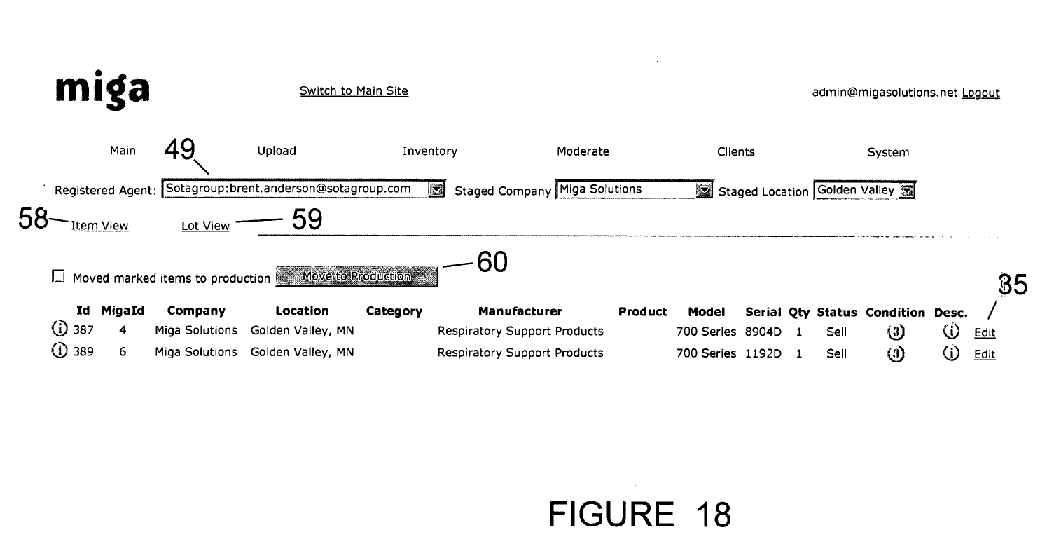

When the administrator clicks on “Staging” button 54, he is taken to the screen shown in FIG. 18. On the FIG. 18 screen, the administrator first selects the registered agent via registered agent drop-down menu 49; selects a particular company among those for which the registered agent manages assets, via staged company drop-down menu 56; and selects a particular location at the selected company, via staged location drop-down menu 57. The pieces of equipment corresponding to those entered parameters is displayed. The administrator can toggle between this item view and a view of the multi-piece lots corresponding to the entered parameters by alternately clicking on “Item View” button 58 and “Lot View” button 59.

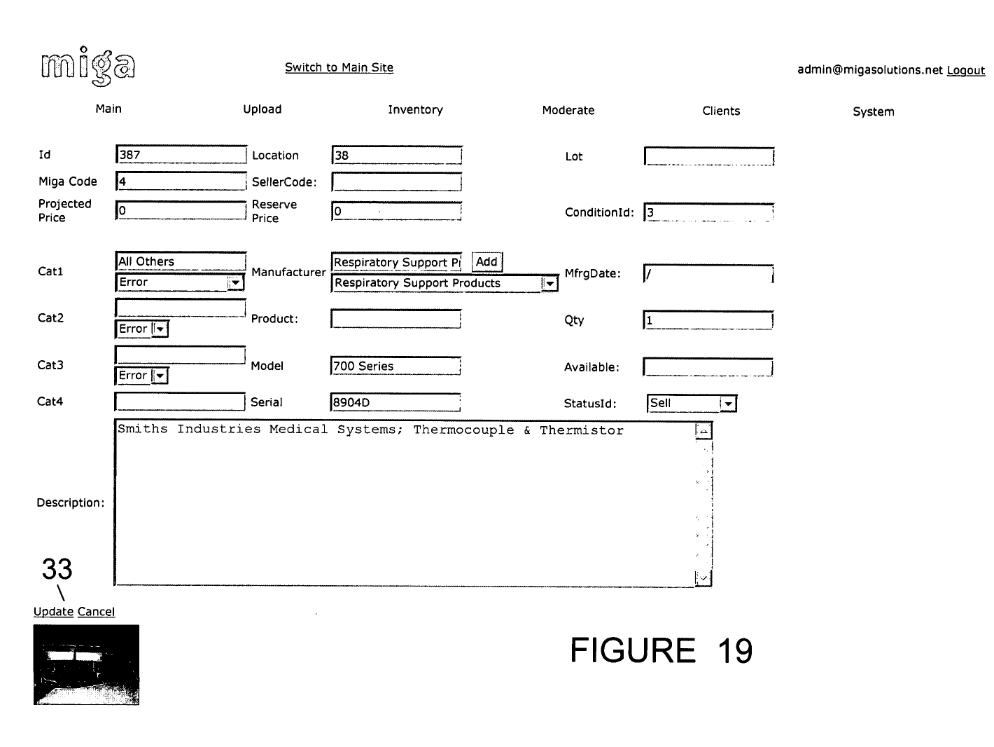

In the FIG. 18 screen, various information about the staged equipment is shown, including ID number, company, location, category/sub-category, manufacturer, product, model name/number, serial number, quantity, status, condition, and a description. The administrator can modify the information about a particular piece of equipment by clicking on “Edit” button 35. The administrator is then taken to the screen shown in FIG. 19, wherein changes to the equipment information may be made and saved via “Update” button 33. When the administrator is satisfied that the equipment information is accurate, he can move items of equipment from the staging area onto the trading platform, by marking/checking the items and clicking on “Move to Production” button 60.

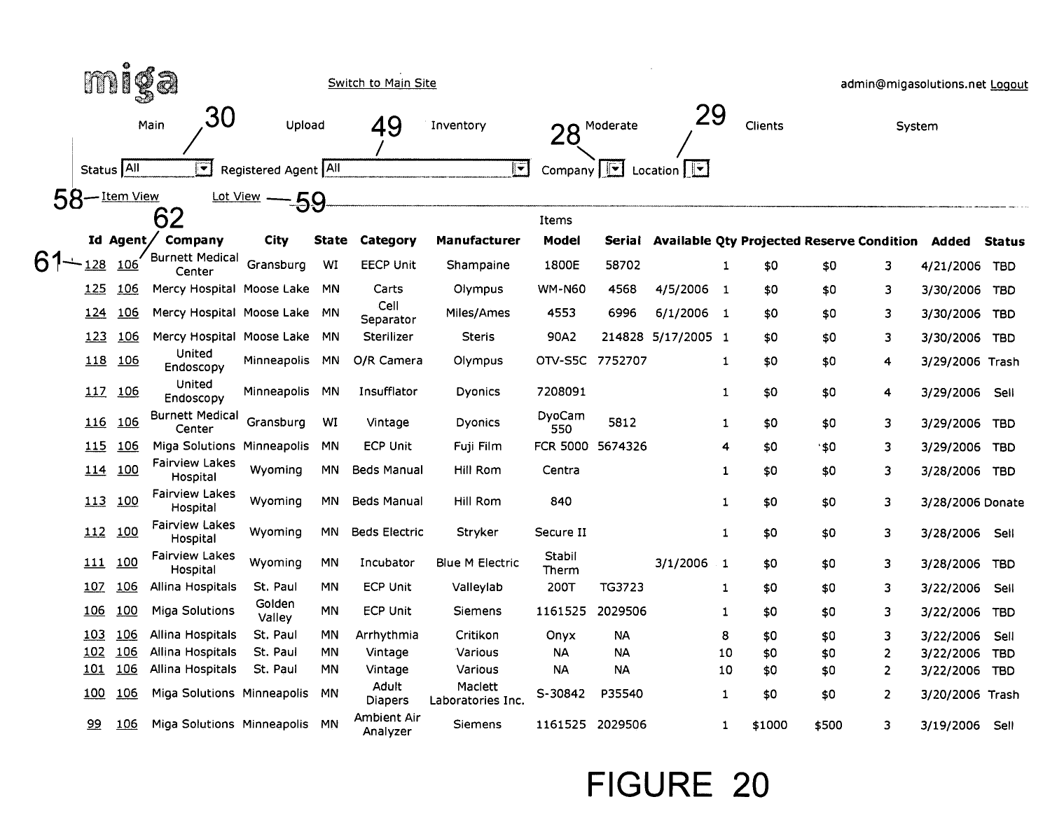

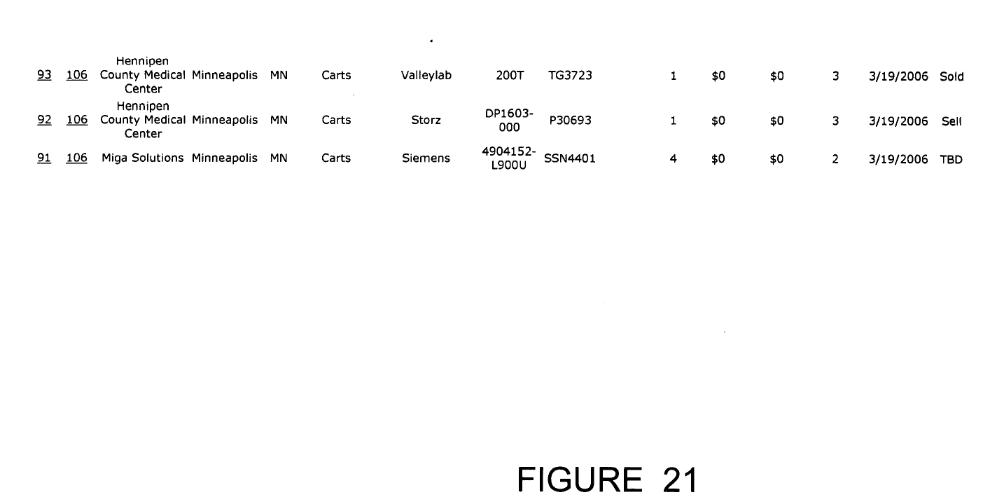

When the administrator clicks on “Manage Inventory” button 55, he is taken to the screen shown in FIGS. 20 & 21, wherein all the equipment active on the trading platform is displayed. The administrator can choose to see all the active equipment or just a portion of the active equipment, by selecting parameters from status drop-down menu 30, registered agent drop-down menu 49, company drop-down menu 28, and location drop-down menu 29. The administrator can also toggle between a view of individual pieces of equipment and a view of multi-piece lots, via “Item View” button 58 and “Lot View” button 59.

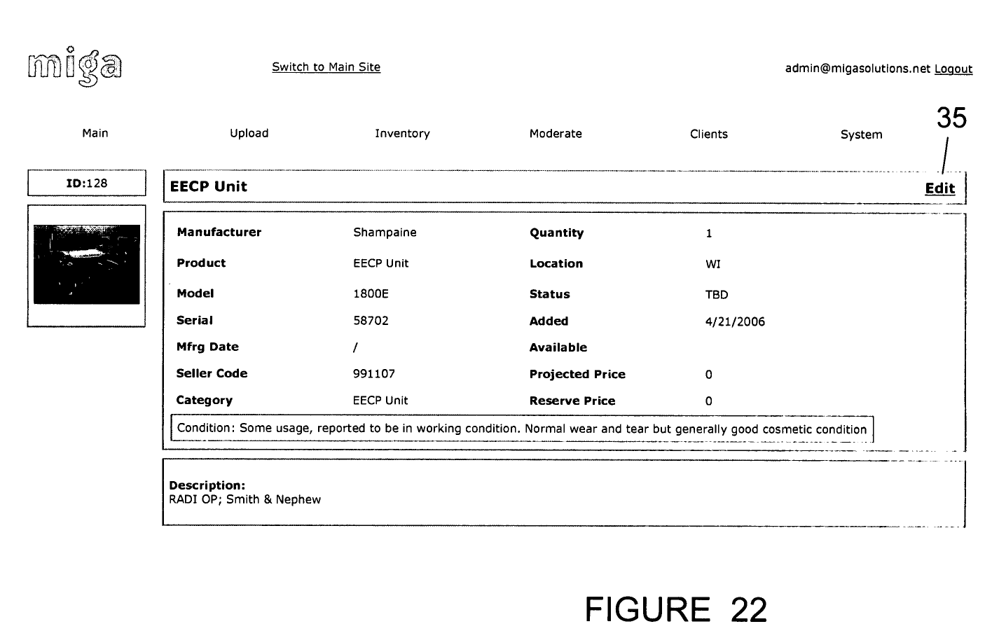

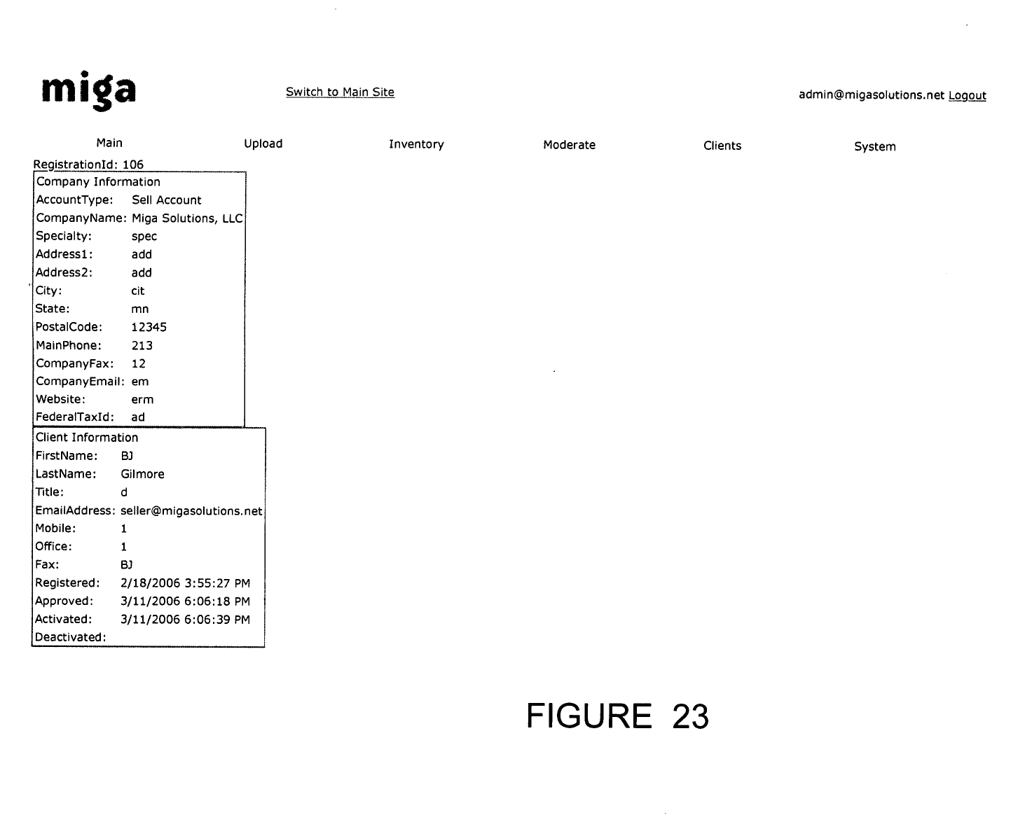

In the item view screen shown in FIG. 20, various information about the equipment is shown, including ID number, registered agent number, company, company location, category/sub-category, manufacturer, model name/number, serial number, date available, quantity, projected and reserve prices, condition, date added to the platform, and status. The administrator can click on “ID” button 61 to be taken to the screen shown in FIG. 22. FIG. 22 displays various information about the piece of equipment, and is similar to the user screen shown in FIG. 5 except that the administrator can edit the equipment information via FIG. 22, by clicking on “Edit” button 35. The administrator can also click on “Agent” button 62 to be taken to the screen shown in FIG. 23. FIG. 23 displays various information about the registered agent associated with the particular piece of equipment.

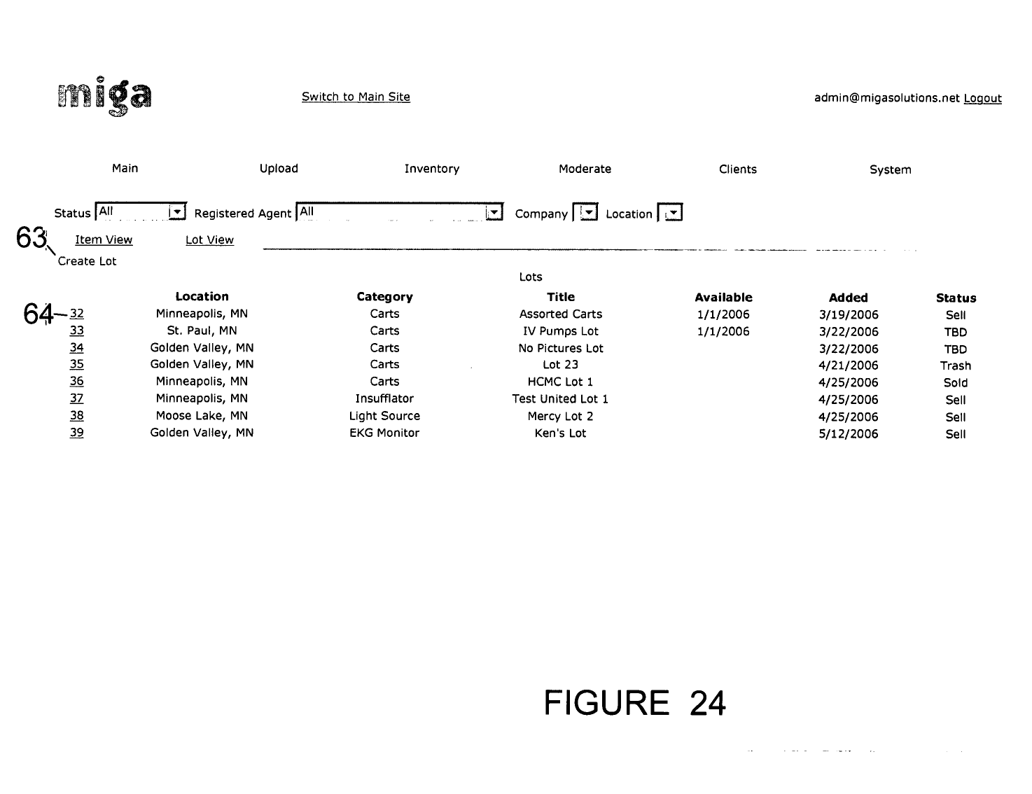

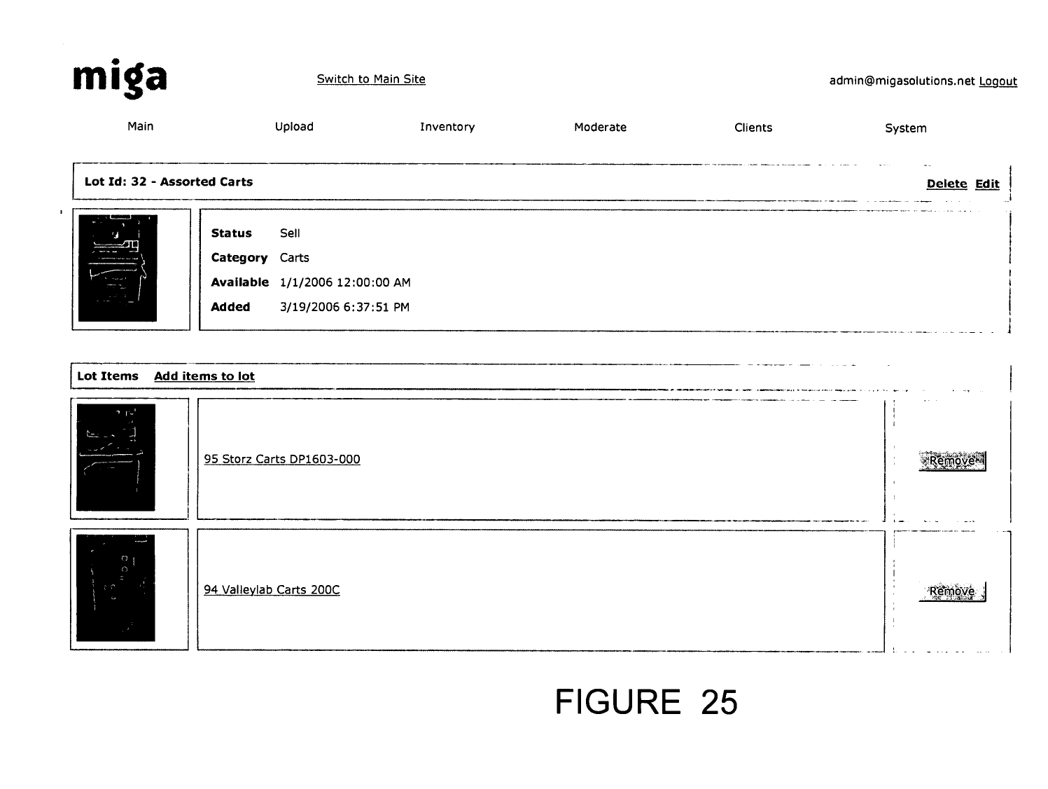

The administrator clicks on “Lot View” button 59 to be taken to the screen shown in FIG. 24. Various information about the active lots is shown in FIG. 24, including Lot ID number, location, company, category/sub-category, lot title, date available, date added to the platform, and status. The administrator can display all the active lots, or a subset of the active lots in a manner similar to that discussed for FIGS. 20 & 21. The administrator can also create a new multi-piece lot of equipment, by clicking on “Create Lot” button 63. The administrator can click on “Lot ID” button 64 to be taken to the screen shown in FIG. 25. FIG. 25 displays various information about the selected lot, including the equipment in the lot. Via the screen shown in FIG. 25, the administrator can modify the lot by adding or deleting equipment, and can also view or modify information about a particular piece of equipment in the lot.

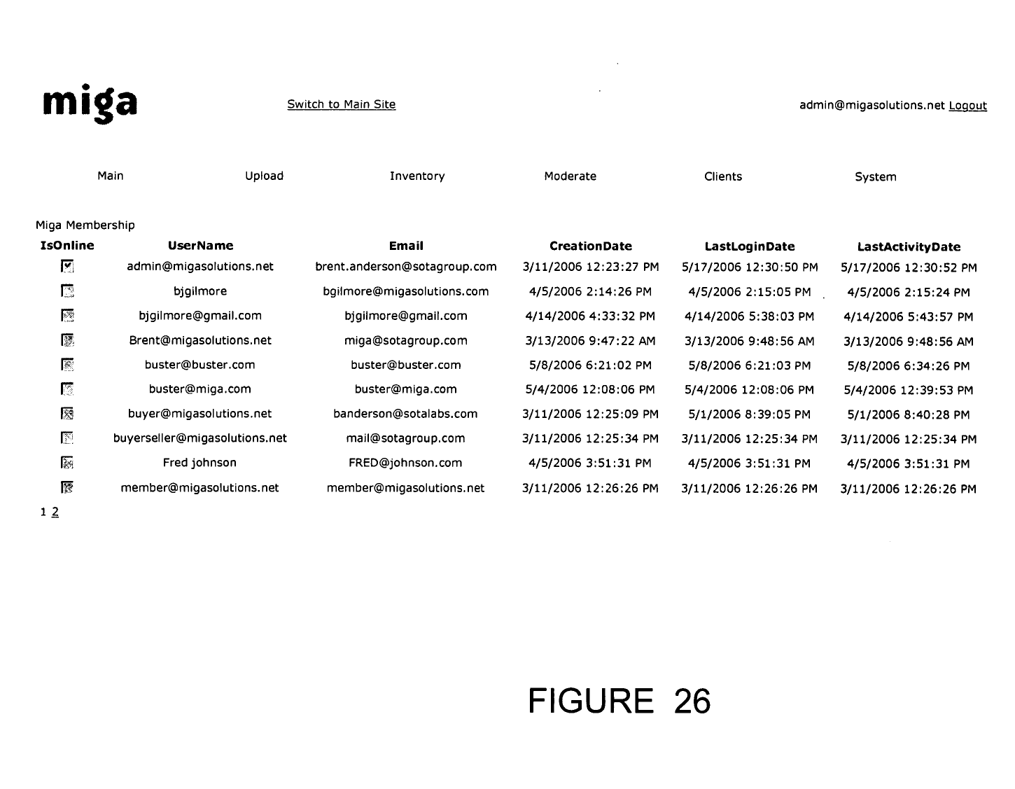

Returning now to FIG. 15, “Moderate” button 44 has six associated sub-selections, reached by moving the cursor over “Moderate” button 44 and making a selection, or via “Members” button 65, “Registered” button 66, “Approved” button 67, “Declined” button 68, “Active” button 69, and “Inactive” button 70. As shown in FIG. 26, clicking on “Members” button 65 allows the administrator to view all the general members who have signed up to view equipment on the trading platform and make offers for equipment.

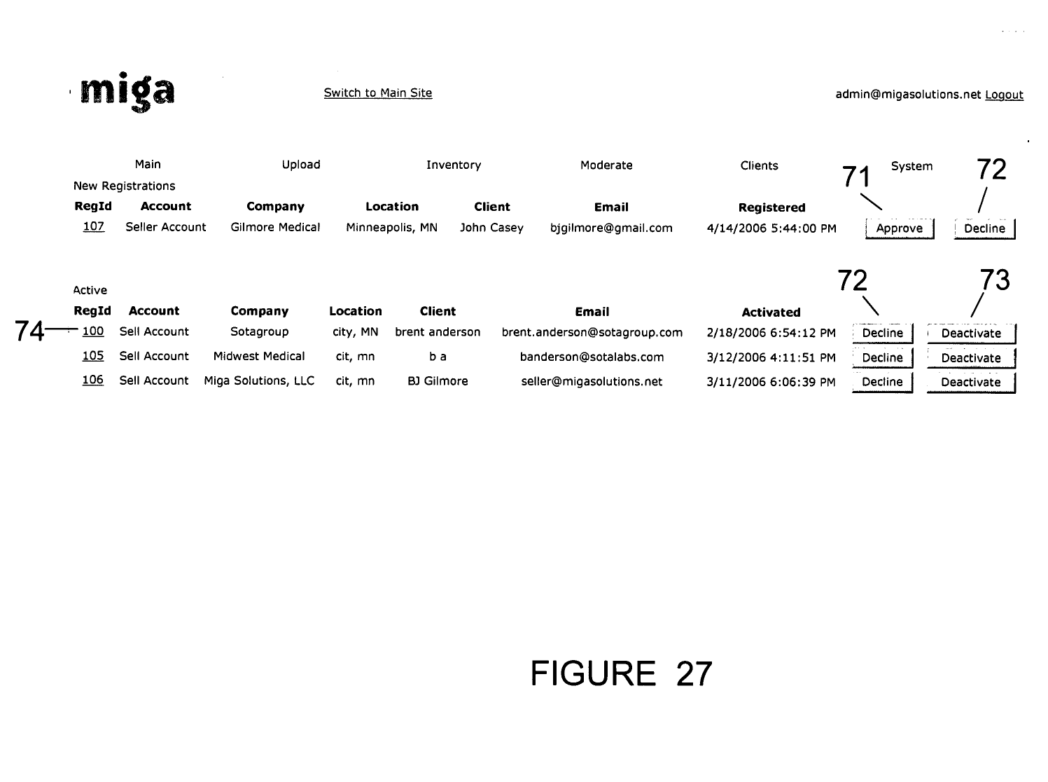

As shown in FIG. 27, clicking on “Registered” button 66 allows the administrator to view all members who have filled out a registration profile to sell on the trading platform. FIG. 27 displays new registrations that have not yet been approved or declined by the administrator, as well as active registrations. Various registration information is displayed, including the registration ID, account, company, location, client, email address, and date and time the registration (i.e., approval to sell on the trading platform) was activated. For new registrations, the date and time the members submitted their registration profile is shown. The administrator can approve or decline a registration by clicking on “Approve” button 71 or “Decline” button 72 respectively, and can also decline or deactivate a previously-approved registration by clicking on “Decline” button 72 or “Deactivate” button 73 respectively. When the administrator approves a registration by clicking on “Approve” button 71, an email is sent to the registered user, informing him of preliminary approval and asking him to consent to being linked to the system. When the registered user returns his consent, approval is official. This “seller verification” part of the system provides buyers with additional assurance that a seller is trusted and allowed to complete transactions, per any regulations that govern transfers of assets including but not limited to medical equipment.

By clicking on “Reg. ID” button 74, the administrator is taken to a screen similar to that shown in FIG. 23, where he can access further information about the registrant. Finally, by clicking on the “Approved” button 67, “Declined” button 68, “Active” button 69, and “Inactive” button 70, the administrator can view and modify previously approved seller registrations, previously declined seller registrations, active seller registrations, and inactive seller registrations respectively.

Returning now to FIG. 15, “Clients” button 45 has one associated sub-selection, reached by moving the cursor over “Clients” button 45 and making a selection, or via “Search” button 75. “Search” button 75 allows the administrator to search clients—i.e., search companies, locations, and contacts that have been added by registered sellers or by the administrator.

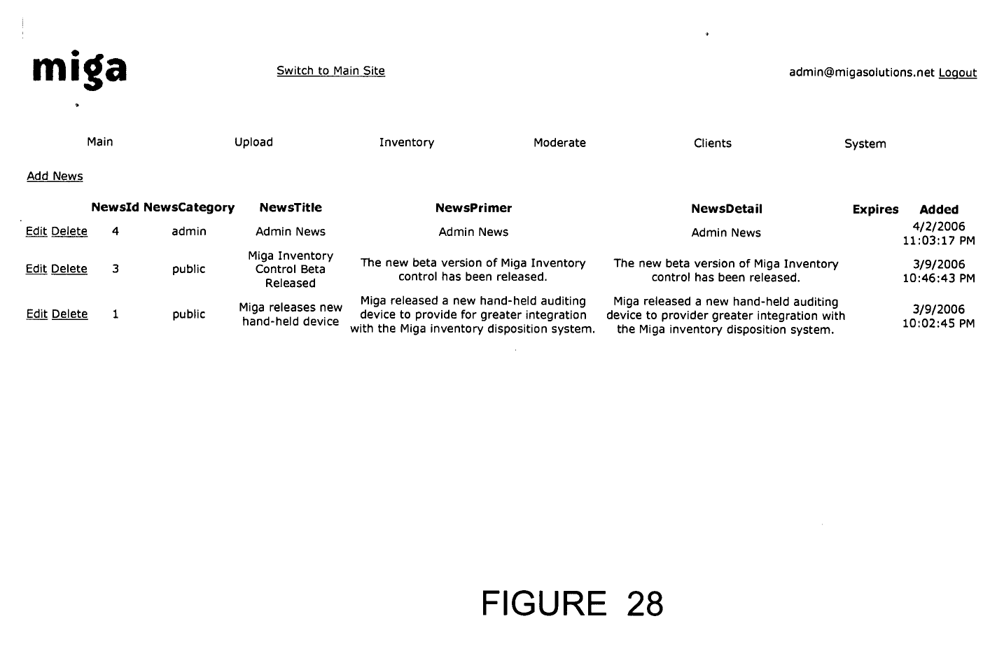

Continuing with FIG. 15, “System” button 46 has two associated sub-selections, reached by moving the cursor over “System” button 46 and making a selection, or via “Messages” button 76 and “News” button 77. “Messages” button 76 allows the administrator to direct messages to individual members or groups of members, or direct messages to members having a certain role. As shown in FIG. 28, “News” button 77 allows the administrator to view previously-added news articles about the system and method that are displayed on the main user page shown in FIG. 2. The administrator can edit a previously-displayed news article, delete it, or add a new article.

While the above descriptions contain many specificities, these shall not be construed as limitations on the scope of the invention, but rather as exemplifications of embodiments thereof. Many other variations are possible without departing from the spirit of the invention. Accordingly, the scope of the invention should be determined not by the embodiments illustrated, but by the appended claims and their legal equivalents.

I/We claim:

1) An asset management system, comprising:
a) a data input module for entering asset data concerning at least one asset, wherein the asset data comprises a plurality of characteristics of the at least one asset, and the data input module links at least two of the plurality of characteristics together into a combined data record for the at least one asset, and
b) a data validation module that receives the asset data entered via the data input module, verifies the entered asset data by comparing the entered asset data against a database containing asset information known to be correct, and modifies the entered asset data to the asset information known to be correct if a difference is found.

2) The system of claim 1, wherein the system also comprises a trading module that receives the asset data validated by the data validation module, and facilitates a transfer of an asset between users of the system.

3) The system of claim 2, wherein a user of the trading module enters information into the system concerning an asset that the user desires but which is not yet available in the system.

4) The system of claim 2, wherein a first user of the trading module redistributes an asset of the first user to a second user in the same organization as the first user.

5) The system of claim 2, wherein a user of the trading module is a non-profit entity, and said user receives an asset donated by another user of the system.

6) The system of claim 2, wherein the entered asset data concerns at least two assets, and the assets are bundled together and offered for a collective price.

7) The system of claim 1, wherein the entered asset data concerns at least one medical equipment asset.

8) The system of claim 1, wherein the data input module is a portable device that transmits the entered asset data to the system.

9) The system of claim 1, wherein the entered asset data includes auditory information concerning the at least one asset, and the auditory information is recorded using said data input module.

10) The system of claim 1, wherein the entered asset data includes visual information concerning the at least one asset, and the visual information is recorded using said data input module.

11) The system of claim 1, wherein the data validation module also compares the entered asset data against applicable federal, state, or local regulations and determines whether those regulations require a further action.

12) A medical equipment management system, comprising:
a) a data input module for entering data concerning at least one piece of medical equipment, wherein the data comprises a plurality of characteristics of the at least one piece of medical equipment, and the data input module links at least two of the plurality of characteristics together into a combined data record for the at least one piece of medical equipment, and
b) a data validation module that receives the medical equipment data entered via the data input module, verifies the entered medical equipment data by comparing the entered medical equipment data against a database containing medical equipment information known to be correct, and modifies the entered medical equipment data to the medical equipment information known to be correct if a difference is found.

13) The system of claim 12, wherein the system also comprises a trading module that receives the medical equipment data validated by the data validation module, and facilitates a transfer of medical equipment between users of the system.

14) The system of claim 13, wherein a user of the trading module enters information into the system concerning medical equipment that the user desires but which is not yet available in the system.

15) The system of claim 13, wherein a first user of the trading module redistributes medical equipment of the first user to a second user in the same organization as the first user.

16) The system of claim 13, wherein a user of the trading module is a non-profit entity, and said user receives medical equipment donated by another user of the system.

17) The system of claim 13, wherein the entered medical equipment data concerns at least two pieces of medical equipment, and the pieces of medical equipment are bundled together and offered for a collective price.

18) The system of claim 12, wherein the data input module is a portable device that transmits the entered medical equipment data to the system.

19) The system of claim 12, wherein the entered medical equipment data includes auditory information concerning the medical equipment, and the auditory information is recorded using said data input module.

20) The system of claim 12, wherein the entered medical equipment data includes visual information concerning the medical equipment, and the visual information is recorded using said data input module.

21) The system of claim 12, wherein the data validation module also compares the entered medical equipment data against applicable federal, state, or local regulations and determines whether those regulations require a further action.

22) A method of managing assets, comprising the steps of:
a) entering, by a data input module, asset data concerning at least one asset, wherein the asset data comprises a plurality of characteristics of the at least one asset, and the data input module links at least two of the plurality of characteristics together into a combined data record for the at least one asset,
b) receiving, by a data validation module, the asset data entered via the data input module,
c) verifying, by the data validation module, the entered asset data by comparing the entered asset data against a database containing asset information known to be correct, and
d) modifying, by the data validation module, the entered asset data to the asset information known to be correct if a difference is found.

23) The method of claim 22, wherein the method also comprises the steps of receiving, by a trading module, the asset data validated by the data validation module, and facilitating, by the trading module, a transfer of an asset between users of the trading module.

24) The method of claim 23, wherein a user of the trading module enters information into the system concerning an asset that the user desires but which is not yet available in the trading module.

25) The method of claim 23, wherein a first user of the trading module redistributes an asset of the first user to a second user in the same organization as the first user.

26) The method of claim 23, wherein a user of the trading module is a non-profit entity, and said user receives an asset donated by another user of the trading module.

27) The method of claim 23, wherein the entered asset data concerns at least two assets, and the assets are bundled together and offered for a collective price.

28) The method of claim 22, wherein the entered asset data concerns at least one medical equipment asset.

29) The method of claim 22, wherein the data input module is a portable device that transmits the asset data after it has been entered.

30) The method of claim 22, wherein the entered asset data includes auditory information concerning the at least one asset, and the auditory information is recorded using said data input module.

31) The method of claim 22, wherein the entered asset data includes visual information concerning the at least one asset, and the visual information is recorded using said data input module.

32) The method of claim 22, wherein the data validation module also compares the entered asset data against applicable federal, state, or local regulations and determines whether those regulations require a further action.

33) A method of managing medical equipment assets, comprising:
a) entering, by a data input module, data concerning at least one piece of medical equipment, wherein the data comprises a plurality of characteristics of the at least one piece of medical equipment, and the data input module links at least two of the plurality of characteristics together into a combined data record for the at least one piece of medical equipment,
b) receiving, by a data validation module, the medical equipment data entered via the data input module,
c) verifying, by the data validation module, the entered medical equipment data by comparing the entered medical equipment data against a database containing medical equipment information known to be correct, and
d) modifying, by the data validation module, the entered medical equipment data to the medical equipment information known to be correct if a difference is found.

34) The method of claim 33, wherein the system also comprises the steps of receiving, by a trading module, the medical equipment data validated by the data validation module, and facilitating, by the trading module, a transfer of medical equipment between users of the trading module.

35) The method of claim 34, wherein a user of the trading module enters information into the system concerning medical equipment that the user desires but which is not yet available in the trading module.

36) The method of claim 34, wherein a first user of the trading module redistributes medical equipment of the first user to a second user in the same organization as the first user.

37) The method of claim 34, wherein a user of the trading module is a non-profit entity, and said user receives medical equipment donated by another user of the trading module.

38) The method of claim 34, wherein the entered medical equipment data concerns at least two pieces of medical equipment, and the pieces of medical equipment are bundled together and offered for a collective price.

39) The method of claim 33, wherein the data input module is a portable device that transmits the medical equipment data after it is entered.

40) The method of claim 33, wherein the entered medical equipment data includes auditory information concerning the medical equipment, and the auditory information is recorded using said data input module.

41) The method of claim 33, wherein the entered medical equipment data includes visual information concerning the medical equipment, and the visual information is recorded using said data input module.

42) The method of claim 33, wherein the data validation module also compares the entered medical equipment data against applicable federal, state, or local regulations and determines whether those regulations require a further action.

43) A medical equipment data validation method, comprising:
a) receiving the medical equipment data,
b) verifying the medical equipment data by comparing the medical equipment data against a database containing medical equipment information known to be correct, and
c) modifying the medical equipment data to the medical equipment information known to be correct if a difference is found.

44) The method of claim 43, wherein the verifying step comprises a series of successive comparisons, and each successive comparison uses the results of the previous comparisons for more efficient verification.

45) The method of claim 43, wherein the medical equipment data validation method also comprises the steps of comparing the medical equipment data against applicable federal, state, or local regulations and determining whether those regulations require a further action.

46) The method of claim 43, wherein the medical equipment data validation method also comprises the steps of comparing the medical equipment data against a database of equipment known to be capable of storing patient information, and alerting that the medical equipment should not be transferred until the patient information has been erased.

47) The method of claim 43, wherein the medical equipment data that is verified includes a name of a manufacturer of the medical equipment.

48) The method of claim 43, wherein the medical equipment data that is verified includes a product name associated with the medical equipment.

49) The method of claim 43, wherein the medical equipment data that is verified includes a model number associated with the medical equipment.

50) The method of claim 43, wherein the medical equipment data that is verified includes a manufacturing date associated with the medical equipment.

51) The method of claim 43, wherein the medical equipment data that is verified includes a serial number associated with the medical equipment.

52) The method of claim 43, wherein the medical equipment data that is verified includes a category classification associated with the medical equipment.

53) A data input system for entering data concerning at least one asset, comprising:
a) a data input module for entering asset data concerning at least one asset, wherein
b) the asset data comprises a plurality of characteristics of the at least one asset, and
c) the data input module links at least two of the plurality of characteristics together into a combined data record for the at least one asset.

54) The system of claim 53, wherein the entered asset data concerns at least one medical equipment asset.

55) The method of claim 54, wherein the data input module is a portable device that transmits the asset data after it has been entered.

56) The system of claim 54, wherein the entered medical equipment data includes auditory information concerning the medical equipment, and the auditory information is recorded using said data input module.

57) The system of claim 54, wherein the entered medical equipment data includes visual information concerning the medical equipment, and the visual information is recorded using said data input module.

58) The system of claim 54, wherein the entered medical equipment data includes a textual description of the medical equipment, and the textual description is entered using said data input module.

59) The system of claim 54, wherein the data input module reads an identifying tag associated with the medical equipment and selected from the group consisting of: bar code, radio frequency identification tag, magnetic tag.

60) The method of claim 54, wherein the medical equipment data that is entered includes a name of a manufacturer of the medical equipment.

61) The method of claim 54, wherein the medical equipment data that is entered includes a product name associated with the medical equipment.

62) The method of claim 54, wherein the medical equipment data that is entered includes a model number associated with the medical equipment.

63) The method of claim 54, wherein the medical equipment data that is entered includes a manufacturing date associated with the medical equipment.

64) The method of claim 54, wherein the medical equipment data that is entered includes a serial number associated with the medical equipment.

65) The method of claim 54, wherein the medical equipment data that is entered includes a category classification associated with the medical equipment.
